(19) United States
(12) Patent Application Publication
(10) Pub. No.: US 2023/0218589 A1
(43) Pub. Date: Jul. 13, 2023

(54) COMPOUND FOR PREVENTION OR TREATMENT OF A SKIN CANCER OR SKIN PRECANCER

(71) Applicant: UNIQUEST PTY LTD, St. Lucia, Queensland (AU)

(72) Inventors: James WELLS, St Lucia, Queensland (AU); Brian William DYMOCK, St Lucia, Queensland (AU); Andrew HARVEY, St Lucia, Queensland (AU); Terrie-Anne COCK, St Lucia, Queensland (AU); Rebecca POUWER, St Lucia, Queensland (AU); Kimberley BEAUMONT, St Lucia, Queensland (AU)

(21) Appl. No.: 18/009,642

(22) PCT Filed: Jun. 9, 2021
(86) PCT No.: PCT/AU2021/050582

§ 371 Date: Dec. 9, 2022

(30) Foreign Application Priority Data
Jun. 9, 2020 (AU) 2020901895

(51) Int. Cl. A61K 31/436; A61P 35/00; A61P 17/00; A61K 9/00

(52) U.S. Cl. CPC A61K 31/436; A61P 35/00; A61P 17/00; A61K 9/0014

(57) ABSTRACT

The present invention relates, inter alia, to a method of preventing or treating skin cancer or skin precancer, the method comprising locally administering to the skin of a subject in need thereof an effective amount of a compound of Formula (I) or a pro-drug thereof, Formula (I) wherein the subject is being administered an immunosuppressant agent that binds to FKBP12. The agent that binds to FKBP12 may be tacrolimus. The invention also relates to methods of preventing or treating a skin condition, disorder or disease associated with administration of an immunosuppressant agent that binds to FKBP12, and to uses of the compound of Formula (I) in treating skin cancer or skin precancer, or a skin condition, disorder or disease associated with administration of an immunosuppressant agent that binds to FKBP12.

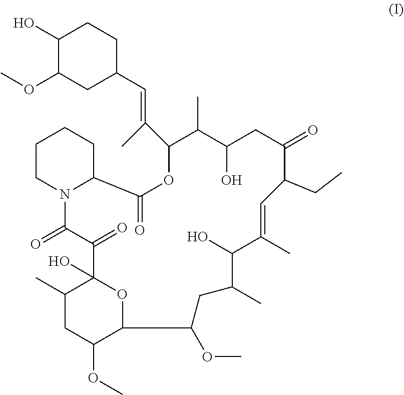 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

### TECHNICAL FIELD

The present invention relates, inter alia, to methods involving local administration of a compound to the skin of a subject for the prevention or treatment of skin cancer, skin precancer and other skin conditions, diseases and disorders, wherein the subject being administered the compound is also being administered immunosuppressive medication. The present invention also relates to uses of the compound and pharmaceutical compositions for local administration to the skin which include the compound.

### BACKGROUND ART

It will be clearly understood that, if a prior art publication is referred to herein, this reference does not constitute an admission that the publication forms part of the common general knowledge in the art in Australia or in any other country.

Organ transplant recipients require life-long immunosuppression in order to prevent their immune systems from rejecting their transplanted organs. Typically, a combination of drugs is taken by patients to prevent organ rejection. There are various classes of immunosuppressive maintenance drugs which are typically taken in combination, including: calcineurin inhibitors such as tacrolimus and cyclosporine A; antiproliferative agents such as mycophenolate mofetil, mycophenolate sodium and azathioprine; mTOR inhibitors such as sirolimus and everolimus; steroids such as prednisone; and antibodies such as basiliximab. Tacrolimus is the primary immunosuppressive agent currently used in the majority of organ transplant patients, and in most cases it is used in combination with other immunosuppressive drugs.

Suppression of the immune system can result in various side effects, including an increased prevalence of cancer. Overall, the standardized incidence ratio (SIR) of all cancers is 2-10 in organ transplant recipients compared with age-matched controls in the general population. Skin cancers are the most common malignancy seen in organ transplant recipients, especially cutaneous squamous cell carcinomas (cSCCs) which have a SIR of up to 198 in organ transplant recipients compared to the general population. Other frequently occurring cancers in the normal population—carcinomas of the bronchus, prostate, colon, rectum and breast, are only slightly increased in organ transplant recipients.

More specifically, patients taking immunosuppressants are at increased risk of developing actinic keratosis (AKs) and cutaneous malignancies (such as cutaneous squamous cell carcinomas (cSCCs)). Furthermore, AKs in organ transplant recipients have an increased chance of developing into invasive cSCCs than in immunocompetent patients (Heppt et al 2019).

The current standard of care for individual cSCCs is cryosurgery or resection. There have been reports of organ transplant recipients on long-term immunosuppressive therapy needing up to 10 surgeries per month to remove cSCCs, including some kidney transplant patients having 120 primary tumours removed each year. Such surgeries are time-consuming, carry risks for the patient (such as failure to excise all of the cancer), and if the patient is elderly it can be more difficult for them to recover from surgeries. Furthermore, many cSCCs are in places such as the face where repeated resection is difficult and limited, and hence there is a clear unmet medical need for new non-surgical treatments.

Skin cancers (such as cSCCs) typically develop from normal skin through various precursors. For example, cancer precursors may include actinic keratoses (AKs) and intraepidermal carcinomas (IECs), which can develop into cSCCs. Furthermore, benign cancers can become malignant. Immunosuppressed patients would typically first develop such cancer precursors as a first step in developing cSCCs, and treatment in these early stages of disease is desirable. Current treatments for AKs include cryosurgery (but this also causes damage to surrounding skin) and topical therapies such as 5-fluorouracil and imiquimod (Aldara cream) (but these can have low clearance rates and considerable side-effects that severely limit their use). The topical therapies are particularly used for organ transplant recipients at risk of field-cancerisation. However, the complete clearance rate of AKs for 5-fluorouracil is 11%, and for imiquimod is 27.5-62.1% (Heppt et al. 2019).

While the foregoing description particularly discusses the prevention and treatment of skin cancers and skin precancers, and particularly AKs and SCCs, the present invention should not be considered to be limited to this use.

### SUMMARY OF INVENTION

The present invention is directed, inter alia, to methods for treating or preventing skin cancer or skin precancer in immunosuppressed patients, or which may provide the consumer with a useful or commercial choice.

With the foregoing in view, the present invention in some forms resides broadly in methods for treating or preventing skin cancer, or other skin (including nail or hair) conditions, disorders or diseases which may result from treatment with immunosuppressive agents.

In a first aspect, the present invention provides a method of preventing or treating skin cancer or skin precancer, the method comprising locally administering to the skin of a subject in need thereof an effective amount of a compound of Formula (I) or a prodrug thereof,

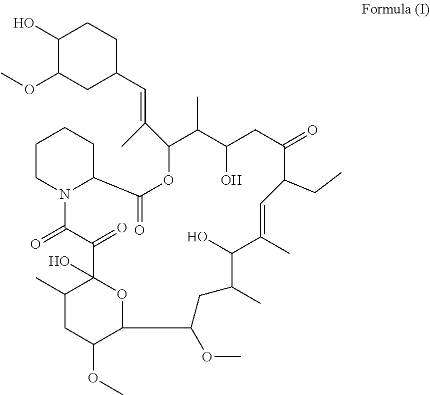 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

wherein the subject is being administered an immunosuppressant agent that binds to FKBP12.

Advantageously, the inventors of the present application have surprisingly found that the compound of Formula (I) can be administered locally to prevent or treat skin cancers caused by use of an immunosuppressant agent that binds to FKBP12 such as tacrolimus.

In one embodiment of the first aspect of the invention, the skin cancer is a cutaneous malignancy, such as a cutaneous squamous cell carcinoma (cSCC), a malignant melanoma, a Merkel cell carcinoma (MCC), or a basal cell carcinoma (BCC). The skin cancer may be a cutaneous malignancy, such as a cutaneous squamous cell carcinoma (cSCC), a malignant melanoma, or a Merkel cell carcinoma (MCC). The skin precancer may be a precancerous skin lesion. The prevention of skin cancer or skin precancer may include cutaneous field cancerisation, or prevention in a region at risk of developing skin cancers or skin precancers. The skin precancer may be an actinic keratosis (AK), an intraepidermal carcinoma (IEC, such as Bowen's disease) or a cutaneous malignancy such as Kaposi' s sarcoma. The skin cancer being prevented or treated may be a malignant or benign cancer. In one embodiment, the skin cancer is especially a cutaneous squamous cell carcinoma (cSCC). In one embodiment, the skin precancer is an actinic keratosis (AK).

In one embodiment, the compound administered is the compound of Formula (I). The compound of Formula (I) includes chiral carbon atoms, and the compound of Formula (I) includes all of the possible stereoisomeric pairs (i.e. the R and S stereochemistry at each chiral carbon atom). The compound of Formula (I) also includes two C═C double bonds, and the Z or E configuration of these bonds is as illustrated in the compound of Formula (I). In one embodiment, the compound of Formula (I) is Compound 1, as shown below:

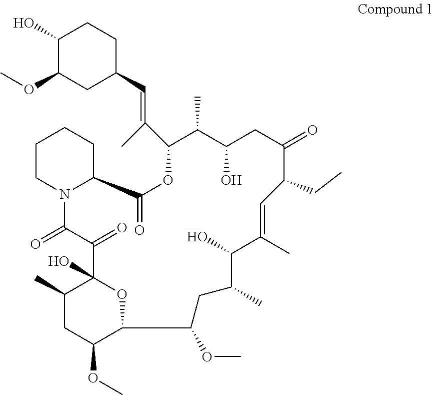 [SMILES: CC[C@@H]1/C=C(\C)[C@@H](O)[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

In one embodiment, the immunosuppres sant agent that binds to FKBP12 is tacrolimus. Tacrolimus (FK506) acts by binding to its cellular target (the protein FKBP12), and this complex binds to and inactivates calcineurin as illustrated in FIG. 1. While the compound of Formula (I) is similar in structure to tacrolimus, a key difference is the presence of a hydroxy group at C-20, and it is consistent with experimental observations that this hydroxy group prevents binding to calcineurin. Below is provided compound 1 in which various ring carbon numbers are marked.

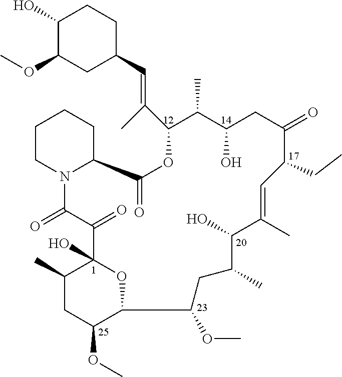 [SMILES: CC[C@@H]1/C=C(\C)[C@@H](O)[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

Compound 1 is disclosed in EP0463690 which relates to various macrolides which are useful as antagonists of FK506 type immunosuppressants. EP0463690 claims a wide range of structurally similar analogues of tacrolimus (FK506). Various administration routes are discussed in this document, but parenteral or oral administration is preferred which would provide a systemic effect. A single assay is discussed in EP0463690 using T lymphocytes isolated from spleens taken from C57B1/6 mice. Dilutions of the compounds were cultured with tacrolimus at a concentration of 1.2 nM (a concentration which inhibits T cell proliferation by 100%). The concentration of the compound required to inhibit tritiated thymidine uptake of T cells by 50% was determined, and the tested compounds were reported to possess an ED50 of <5×10⁻⁵ M. However, EP043690 only discusses an effect of compound 1 on T cells at a potentially high concentration (<5×10⁻⁵ M), and this is distinct from the subject matter of the present invention as outlined further below.

Furthermore, and as outlined below, the biological interactions which would result in the treatment or prevention of skin cancer or skin precancers through the local application of a compound of Formula (I) to the skin are very complex and extremely difficult to predict. Much of the discussion below relates to the use of tacrolimus as an immunosuppressant, but given that sirolimus (rapamycin) and everolimus both also bind to FKBP12 the inventors believe that the invention is also applicable when these immunosuppressants (and others that bind to FKBP12) are used.

### cSCC Initiation and Progression

The exact causes of cutaneous squamous cell carcinoma (cSCC) initiation and progression in organ transplant recipients are unclear, and the particularly high incidence of cSCCs in organ transplant recipients is unexpected and cannot be fully explained by immunosuppression alone. Overall, the standardized incidence ratio (SIR) of all cancers is 2-10 in organ transplant recipients compared with age-matched controls in the general population (Villeneuve et al. 2007; Engels et al. 2011; Piselli et al. 2013; Tessari et al. 2013; Krynitz et al. 2013; Ekström, Riise, and Tanash 2017; Collett et al. 2010; Vajdic et al. 2006; Cheung et al. 2012; Li et al. 2012; K.-F. Lee et al. 2016). Other frequently occurring cancers in the normal population—carcinomas of the bronchus, prostate, colon, rectum and breast, are only slightly increased in organ transplant recipients (Penn 2000). Skin cancers are the most common malignancy seen in organ transplant recipients, especially cSCC which have a SIR of up to 198 in organ transplant recipients compared to the general population (Krynitz et al. 2013; Moloney et al. 2006; Rizvi et al. 2017; Tessari et al. 2013). Actinic keratosis (AKs), which are transformed keratinocytes that can progress to cSCC (Berman and Cockerell 2013), also have a very high prevalence in organ transplant recipients compared to the general population with a prevalence of over 80% of kidney and liver transplant patients (Flohil et al. 2013; Iannacone et al. 2016).

Immunosuppression and loss of immunosurveillance is likely to contribute to cSCC development in organ transplant recipients, as other immunosuppressed patients such as HIV patients or patients with chronic lymphocytic leukemia (CLL) also have a moderately increased incidence of cSCC (Engels 2019). However, the incidence of cSCC is much higher in organ transplant recipients (SIR of up to 198) than in immunocompromised HIV patients (SIR of 4 (Wheless et al. 2014; Engels 2019)) or CLL patients (SIR of 2-8 (Levi et al. 1996; Ishdorj et al. 2019)). The much higher incidence of cSCC in organ transplant recipients compared to other immunosuppressive disorders suggests there are mechanisms other than just reduced immunosurveillance contributing to cSCC in organ transplant recipients.

In addition to immunosuppression, other risk factors like oncogenic viral infections (J. Wang et al. 2014), UV light exposure, skin pigmentation (Euvrard, Kanitakis, and Claudy 2003) and tumorigenic effects of tacrolimus on keratinocytes (Ming et al. 2015; Canning et al. 2006; Wu et al. 2010; Lan et al. 2007) may synergistically contribute to the development of cSCCs in organ transplant recipients, however the interdependence and relative contributions of these cofactors is still unknown, impeding the development of treatment options (Harwood et al. 2017). Human papillornavirus (HPV) infections are found in up to 90% of SCCs from organ transplant recipients and may contribute to the risk of organ transplant recipients developing cSCC, although the evidence is not conclusive (J. Wang et al. 2014; Aldabagh et al. 2013). Because the interdependence and relative contributions of the different cofactors are not known, it is extremely difficult to anticipate the effect of local tacrolimus antagonism on cSCCs and AKs. Moreover, the effects of tacrolimus on T cells are reversible (Laskin et al. 2017), but it is not known if the effects of tacrolimus on the multiple drivers of cSCCs in organ transplant recipients are also reversible. It is therefore surprising that local tacrolimus antagonism could be a treatment for AKs and cSCCs.

### Tacrolimus has Complex Pro- and Anti-Tumorigenic Effects on Multiple Cell Types

Tacrolimus has effects on conventional T cells, as well as on other immune and non-immune cell types, and these can have pro- and anti-tumorigenic effects on the development and progression of cSCCs. In T cells, tacrolimus mediates its immunosuppressive effect by inhibiting IL-2, IL-3, IL-4, TNFα and IFNγ production, activation and proliferation of T cells (Thomson, Bonham, and Zeevi 1995; Sigal and Dumont 1992; Ruzicka, Assmann, and Homey 1999). The effect of tacrolimus on regulatory T cells (Tregs) has been less clear, as it has mostly been reported to induce proliferation (Kogina et al. 2009; Z. Wang et al. 2009), but also to inhibit or not affect the proliferation of regulatory T cells (Calvo-Turrubiartes et al. 2009; Z. Wang et al. 2009). Tregs inhibit effector immune responses and thus clearance of tumours (Bottomley et al. 2019). Accordingly, and especially in light of the conflicting evidence of the effects of tacrolimus on regulatory T cells, it is surprising that tacrolimus antagonism could assist in clearing tumours.

Other immune cells that are affected by tacrolimus and are known to be involved in tumour biology include B cells (Chung et al. 2014; Traitanon et al. 2015; Glynne et al. 2000), epidermal dendritic cells and Langerhans cells (Panhans-Groll et al. 2001; Wollenberg et al. 2001).

In B cells, tacrolimus inhibits, for example, cell proliferation (Glynne et al. 2000) and IL-10 production (Chung et al. 2014). IL-10 produced by B cells was suggested to be a tumour-promoter of skin carcinogenesis (Schioppa et al. 2011), which could suggest a tumour-supressing effect of tacrolimus by reducing IL-10 production in B cells. It was long believed that B cells have mainly anti-tumour effects, however tumour-promoting roles of B cells have been revealed more recently (Sarvaria et al. 2017). Therefore, it is unclear if the effects of tacrolimus on B cells in cSCC are tumour promoting or supressing.

In the epidermis, tacrolimus has been shown to reduce the stimulatory activity of epidermal dendritic cells and Langerhans cells on T cells (Panhans-Groß et al. 2001; Wollenberg et al. 2001). Epidermal dendritic cells and Langerhans cells have been shown to promote tumour progression in cSCC (Modi et al. 2012; Lewis, Bügler, Fraser, et al. 2015; Lewis, Burgler, Freudzon, et al. 2015; Ravindran et al. 2014), but Langerhans cells have also been shown to decrease tumour growth (Ortner et al. 2017). Therefore, it is not clear if the effect of tacrolimus on epidermal dendritic cells and Langerhans cells is tumour promoting or suppressing.

Tacrolimus also has effects on non-immune cell types which may be tumour-promoting or tumour suppressing. For example, although tacrolimus has been suggested to promote keratinocyte tumour formation (Wu et al. 2010), it has also been shown to inhibit keratinocyte proliferation by arresting the cell cycle at G0/G1 phase which would suggest anti-tumorigenic effects of tacrolimus on keratinocytes (Karashima et al. 1996). However, two other studies showed that tacrolimus had no effect on keratinocyte proliferation (Duncan 1994; Kaplan et al. 1995). Tacrolimus has also been shown to impair UV-induced apoptosis and DNA damage repair (Ming et al. 2015; Canning et al. 2006). Therefore, it is not clear which effect local tacrolimus antagonism will have on keratinocytes in AKs and on cSCC cells in organ transplant recipients especially in the context of the tumour-promoting and tumour-suppressing effects that tacrolimus has on different immune cells also present in the cSCCs. The effects of tacrolimus on T cells are reversible (Laskin et al. 2017), but it is not known if the tumour promoting and supressing effects of tacrolimus on other cell types are also reversible.

Because tacrolimus potentially has tumour-promoting as well as tumour-supressing effects on Tregs, B cells, epidermal dendritic cells, keratinocytes and Langerhans cells it was not clear what effect local tacrolimus antagonism will have on AKs and cSCCs.

### Complexity of the Tumour Immune Microenvironment

The tumour immune microenvironment is complex and may alter patient outcomes or responses to immunotherapies. Transplant-associated cSCC have a high number of suppressive immune cell sub-populations and whether antagonism of tacrolimus can overcome this immunosuppressive tumour microenvironment to induce tumour clearance was unclear, let alone local treatment by a compound of Formula (I) to the skin.

Tumour infiltrating lymphocytes (TILs) are immune cells which comprise many different sub-populations of which some can help clear tumours by directly killing tumour cells, but some can also promote tumorigenesis. The presence of TILs generally correlates with positive cancer outcomes. However, there remain inconsistencies and controversies over the prognostic value of particular sub-populations (Shang et al. 2015). The type of tumour, tumour stage, the location, type and density of particular TILs present as well as their activation state may explain some of the inconsistencies between prognostic studies (Barnes and Amir 2017).

Differences in TIL sub-populations may explain why immunotherapy fails or succeeds in different individuals with the same cancer (Gnjatic et al. 2017; Binnewies et al. 2018; Badalamenti et al. 2019). Surprisingly, some cancer patients even see an acceleration of tumour growth (hyperprogressive disease) after checkpoint inhibitor immunotherapy treatment (Champiat et al. 2017). Increased numbers of regulatory T cells (Tregs) have been linked to failure of immunotherapy (Taylor et al. 2017) as well as hyperprogressive disease (Kamada et al. 2019). Differences in the immune microenvironment may also underlie the lack of initial efficacy of the immune modulator imiquimod in some AK patients as well as AK recurrence after an initial clearance (P. K. Lee et al. 2005), although little is known about imiquimod mode of action (Hanna, Abadi, and Abbas 2016).

In cSCC in immunocompetent patients, several TIL sub-populations have been described with both anti-tumour and pro-tumour functions. CD8+ Cytotoxic T cells (CTLs) can directly kill tumour cells via cytotoxic granules and cytokines. CD8+ T cells have an anti-tumour role in cSCC animal models (Black et al. 2005; Freeman et al. 2014; Nasti et al. 2011; Yusuf et al. 2008), but can also have pro-tumourigenic effects (Daniel et al. 2003). These inconsistencies may be due to modified sub-populations of CD8+ T cells in the tumour microenvironment (Maimela, Liu, and Zhang 2018). For example, a specific CD8+ T cell subset (T-pro) has been identified with cSCC tumour-promoting effects in a mouse model (Roberts et al. 2007). In addition, CD4+ regulatory T cells (Tregs) are known to suppress CD8+T cell activity and contribute to CD8+ T cell exhaustion in tumours including cSCC [(Lai et al. 2016) and reviewed in Crispin and Tsokos 2020]. It is not sufficient just to have CD8+ T cells present for tumour clearance, the CD8+ sub-populations present and the ratio of CD8+ T cells: Tregs are important. High CD8+ T cells: Tregs ratios favour anti-tumour responses, and the inverse favour tumour growth and poor outcomes in many cancers including cSCC (Quezada et al. 2011; Azzimonti et al. 2015).

Transplant-associated cSCC has altered TIL sub-populations compared to immune competent cSCC. In transplant-associated cSCC the majority of studies indicate there is a decreased CD8+:Treg ratio compared to immune-competent cSCC (Strobel et al. 2018; Carroll et al. 2010; Zhang et al. 2013). Markers of exhausted CD8+ T cells (Feldmeyer et al. 2016), which have reduced anti-tumour function (Crispin and Tsokos 2020) have been identified in transplant-cSCCs. In addition, senescent (irreversible cell cycle arrest), terminally differentiated CD8+ T cells in the peripheral blood of organ transplant recipients have been linked to an increased cSCC risk in organ transplant recipients (Bottomley, Harden, and Wood 2016). Notably, it has been shown that once T cells reach a threshold level of exhaustion, they are unable to be rescued (Philip et al. 2017). This indicates that transplant-associated cSCC CD8+ T cells may be dysfunctional, and that this phenotype might not be reversible.

There are several other TIL sub-populations described in cSCC. CD4+ T cells can either promote or inhibit anti-tumour responses (Kim and Cantor 2014). CD4+ Tregs are known to have immune-suppressive function and are associated with more aggressive cSCC tumours (Lai et al. 2016; Kambayashi, Fujimura, and Aiba 2013; Azzimonti et al. 2015). Little is known about the specific role of some of the sub-populations of CD4+ cells in cSCC such as Th9, Th17, Tfh (reviewed in Bottomley et al. 2019). B Cells, dendritic cells, macrophages, myeloid derived suppressor cells and natural killer or innate lymphoid cells may also influence cSCC biology (reviewed in Bottomley et al. 2019). In addition, the role of these TIL sub-populations in transplant-cSCC is not known. The diversity in TIL sub-populations and function in cSCC biology, as well as the unknown functions of some TILs and their roles in transplant-cSCC, makes the impact of tacrolimus antagonism on the tumour immune response hard to predict.

Other changes in transplant-cSCC include decreased IFNγ+ CD4+ Th1 cells (Zhang et al. 2013), decreased antigen-presenting plasmacytoid dendritic cells (Mühleisen et al. 2009), decreased B Cells (Strobel et al. 2018), as well as significantly increased circulating myeloid-derived suppressor cell frequencies (Hock et al. 2012). In addition, it has been suggested that the immune microenvironment in peri-tumoral skin in transplant patients is abnormal (Kosmidis et al. 2010). These changes could also potentially be associated with cSCC development or progression and the effect of tacrolimus antagonism on these changes is also not known.

Although tacrolimus antagonism could be expected to allow recovery of conventional T cell proliferation and activation (Laskin et al. 2017), whether this would be sufficient to revert senescent or exhausted CD8+ T cells back to normal cytotoxic function, or whether it would be sufficient to overcome the immunosuppressive forces of the large number of Tregs present in transplant-cSCC to allow tumour clearance was not clear.

### Calcineurin (the Target of Tacrolimus) has Both Tumour Promoting and Tumour Supressing Functions

Tacrolimus inhibits T cell activity by inhibiting calcineurin mediated activation of the transcription factor Nuclear factor of activated T-cells (NFAT), which is a key regulator of cytokine expression during immune responses (Ruzicka, Assmann, and Homey 1999). The target of tacrolimus-FKBP12 complex, calcineurin, is a well-studied tumour promoter in many different cancer types including colorectal cancer (Peuker et al. 2016; Masuo et al. 2009), breast cancer (Jauliac et al. 2002; Tran Quang et al. 2015; Siamakpour-Reihani et al. 2011), glioblastoma (Brun et al. 2013; Urso et al. 2019; Tie et al. 2013), lymphoblastic leukemia (Gachet et al. 2013; Medyouf et al. 2007), melanoma (Shoshan et al. 2016), lung metastasis (Minami et al. 2013), ovarian cancer (Xu et al. 2016), hepatocellular carcinoma (S. Wang et al. 2012), prostate cancer (Manda et al. 2016), pancreatic cancer (Buchholz et al. 2006) and cervix carcinoma (Huang et al. 2008).

Calcineurin promotes tumours by promoting tumour angiogenesis (Baek et al. 2009; Siamakpour-Reihani et al. 2011), tumour invasion (Jauliac et al. 2002; Tran Quang et al. 2015; Tie et al. 2013), metastasis (Shoshan et al. 2016; Minami et al. 2013) and tumour cell proliferation (Buchholz et al. 2006; S. Wang et al. 2012; Urso et al. 2019).

Calcineurin inhibitors like cyclosporin A and tacrolimus have been shown to reduce tumour cell proliferation (Masuo et al. 2009; Buchholz et al. 2006; Siamakpour-Reihani et al. 2011), reduce invasion (Tie et al. 2013) and even to induce apoptosis and tumour clearance (Medyouf et al. 2007) and have therefore been suggested as potential therapeutic treatments for e.g. breast cancer (Siamakpour-Reihani et al. 2011), bladder cancer (Kawahara et al. 2015), glioblastoma multiforme (Tie et al. 2013) and leukemia (Medyouf et al. 2007).

Given the tumour promoting role of calcineurin in many different cancers, the increased incidence of cSCCs in organ transplant recipients, many of whom are taking calcineurin inhibitors, is surprising. In cSCC mouse models, calcineurin has been shown to suppress the development of tumours (Wu et al. 2010; 2018; Horsley et al. 2008), however its downstream target NFAT1 has also been shown to promote cSCCs (Goldstein et al. 2015; Tripathi et al. 2014), which suggests that calcineurin could also be a tumour-promoter in cSCC.

Due to the tumour-promoting and tumour-supressing effects of calcineurin, the anti-tumourigenic effect of local tacrolimus antagonism (and thus reactivation of calcineurin) in cSCC was surprising. In addition, it has been shown that tumour cells from cyclosporin A treated mice retained their aggressive phenotype, even after cessation of cyclosporin A treatment (Walsh et al. 2011). Therefore, it is surprising that local tacrolimus antagonism would be able to affect the progression of already developed cSCCs, and the anti-tumourigenic effect on cSCCs from antagonism by the compound of Formula (I) to reactivate calcineurin was surprising.

### Topical Tacrolimus is Not Pro-Tumorigenic

As topical tacrolimus is not pro-tumourigenic, even if an association between skin cancer incidence and tacrolimus use was known, it would seem that the increase in skin cancer incidence (and especially cSCCs) would be caused by systemic tacrolimus exposure. Consequently, it is counterintuitive that local tacrolimus antagonism could counteract the systemic tacrolimus effects on AKs and cSCCs in organ transplant recipients.

Despite the evidence indicating a role for tacrolimus in promoting cancer development in organ transplant recipient patients—surprisingly, case-control studies revealed that topical calcineurin inhibitors (tacrolimus or pimecrolimus) are not associated with an increased risk of cSCC in adults (Margolis, Hoffstad, and Bilker 2007; Naylor et al. 2005).

In a mouse model topical tacrolimus application surprisingly drastically reduced chemically (TPA+DMBA) induced tumour formation by 80% (Jiang et al. 1993), whereas another study showed that topical tacrolimus increases the number of skin papillomas, but not cSCCs (Niwa, Terashima, and Sumi 2003).

A 104-week epidermal carcinogenicity study in mice showed no association of skin tumours with daily topical tacrolimus doses up to 260× of the recommended human dose (Protopic® prescribing information).

This suggests that the systemic tacrolimus exposure is causing cSCCs in organ transplant recipients and therefore it would seem counterintuitive that local tacrolimus antagonism could counteract the systemic tacrolimus effects on skin cancer (especially AKs and cSCCs) in organ transplant recipients, let alone local tacrolimus antagonism through local application of a compound of Formula (I) to the skin.

Further features of the first aspect of the present invention are discussed below.

The compound of Formula (I) has asymmetric centres and therefore is capable of existing in more than one stereoisomeric form. The compound of Formula (I) therefore may be in substantially pure isomeric form at one or more asymmetric centres, as well as mixtures, including racemic mixtures thereof. Such isomers may be prepared using chiral reagents, chiral starting materials or intermediates (including natural products), or by chiral resolution. The compound of formula (I) accordingly may be racemic, or may be administered in an enantiomeric excess (such as greater than 50%, 55%, 60%, 65%, 70%, 75%, 80%, 85%, 90%, 95%, 97% or 99%) or diastereomeric excess (such as greater than 50%, 55%, 60%, 65%, 70%, 75%, 80%, 85%, 90%, 95%, 97% or 99%).

The term “prodrug” is used in its broadest sense and encompasses those derivatives that are converted in vivo to a compound of Formula (I). A prodrug may include modifications to one or more of the functional groups of the compound of Formula (I). A prodrug may have the potential to form acidic or basic salts, and the term “prodrug” may include pharmaceutically acceptable salts of the prodrug.

A derivative which is capable of being converted in vivo as used in relation to another functional group includes all those functional groups or derivatives which upon administration into a mammal (such as a human) may be converted into the stated functional group. Those skilled in the art may readily determine whether a group may be capable of being converted in vivo to another functional group using routine enzymatic or animal studies. A prodrug of a compound of Formula (I) may include, for example, an ester or ether of a —OH group of the compound of Formula (I); especially an optionally substituted alkyl ester or optionally substituted alkyl ether; more especially an optionally substituted C₁₋₁₂alkyl ester or an optionally substituted C₁₋₁₂alkyl ether. Such optional substituents may include, for example, one or more of: —NH₂, —NH-alkyl-N(alkyl)₂, —COOH, sulfonyl, nitro, halo, aryl, cycloalkyl, alkyl, heteroaryl, heterocyclyl, and —OH.

The term “cycloalkyl” refers to a saturated or partially unsaturated non-aromatic cyclic hydrocarbon. The cycloalkyl ring may include a specified number of carbon atoms. For example, a 3 to 8 membered cycloalkyl group includes 3, 4, 5, 6, 7 or 8 carbon atoms. The cycloalkyl group may be monocyclic, bicyclic or tricyclic. Where appropriate, the cycloalkyl group may have a specified number of carbon atoms, for example, C₃-C₆ cycloalkyl is a carbocyclic group having 3, 4, 5 or 6 carbon atoms. Non-limiting examples may include cyclopropyl, cyclobutyl, cyclopentyl, cyclopentenyl, cyclohexyl, cyclohexenyl, cyclohexadienyl and the like. Cycloalkyl groups may include a carbonyl group, in which the carbon of the carbonyl group forms part of the ring.

The term “aryl” refers to an aromatic carbocyclic substituent, as commonly understood in the art. It is understood that the term aryl applies to cyclic substituents that are planar and comprise 4n+2π electrons, according to Hückel's Rule. Aryl groups may be monocyclic, bicyclic or tricyclic. Examples of aryl groups include, but are not limited to, phenyl and naphthyl.

The term “heterocyclic” or “heterocyclyl” as used herein, refers to a cycloalkyl group in which one or more carbon atoms have been replaced by heteroatoms independently selected from N, S and O. For example, between 1 and 4 carbon atoms in each ring may be replaced by heteroatoms independently selected from N, S and O. The heterocyclyl group may be monocylic, bicyclic or tricyclic in which at least one ring includes a heteroatom. The heterocyclyl group may include a carbonyl group, in which the carbon of the carbonyl group forms part of the ring. Each of the rings of a heterocyclyl group may include, for example, between 5 and 7 atoms. Examples of heterocyclyl groups include tetrahydrofuranyl, tetrahydrothiophenyl, pyrrolidinyl, 2-pyrrolidonyl, pyrrolinyl, dithiolyl, 1,3 -dioxanyl, dioxinyl, piperidinyl, piperazinyl, morpholinyl, thiomorpholinyl, pyranyl, 1,4-dithiane, piperazin-2,5-dione and decahydroisoquinoline. In a bicyclic or tricyclic heterocyclyl group, one of the rings may be aromatic but not all rings are aromatic.

The term “heteroaryl”, as used herein, refers to a monocyclic, bicyclic or tricyclic ring of up to 7 atoms in each ring, wherein all rings are aromatic and at least one ring contains from 1 to 4 heteroatoms selected from the group consisting of O, N and S. When more than one ring is present the ring is fused. The heteroaryl group may also include a carbonyl group, in which the carbon of the carbonyl group forms part of the ring. Consideration must be provided to tautomers of heteroatom containing ring systems containing carbonyl groups, for example, when determining if a ring is a heterocyclyl or heteroaryl ring. Examples of heteroaryl include thiophene, benzothiophene, benzofuran, benzimidazole, benzoxazole, benzothiazole, benzisothiazole, furan, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, and phenoxazine.

The term “alkyl” refers to a straight-chain or branched alkyl substituent containing from, for example, 1 to about 12 carbon atoms, preferably 1 to about 8 carbon atoms, more preferably 1 to about 6 carbon atoms, even more preferably from 1 to about 4 carbon atoms. Examples of suitable alkyl groups include, but are not limited to, methyl, ethyl, propyl, isopropyl, n-butyl, sec-butyl, isobutyl, tert-butyl, pentyl, isoamyl, 2-methylbutyl, 3-methylbutyl, hexyl, heptyl, 2-methylpentyl, 3 -methylpentyl, 4-methylpentyl, 2-ethylbutyl, 3-ethylbutyl, octyl, nonyl, decyl, undecyl, dodecyl and the like. The number of carbons referred to relates to the carbon backbone and carbon branching but does not include carbon atoms belonging to any substituents, for example the carbon atoms of an alkoxy substituent branching off the main carbon chain.

As used herein, “halo” refers to a halogen atom, especially, F, Cl or Br; more especially F or Cl; most especially F.

Pharmaceutically acceptable salts of such prodrugs includes those salts that are toxicologically safe for local administration to the skin, such as salts prepared from pharmaceutically acceptable non-toxic bases or acids including inorganic or organic bases and inorganic or organic acids. The pharmaceutically acceptable salts may be selected from the group including alkali and alkali earth, ammonium, aluminium, iron, amine, glucosamine, chloride, sulphate, sulphonate, bisulphate, nitrate, citrate, tartrate, bitarate, phosphate, carbonate, bicarbonate, malate, maleate, napsylate, fumarate, succinate, acetate, benzoate, terephthalate, palmoate, piperazine, pectinate and S-methyl methionine salts and the like.

As used herein, the terms “treatment” (or “treating”) and “prevention” (or “preventing”) are to be considered in their broadest contexts. For example, the term “treatment” does not necessarily imply that a patient is treated until full recovery. The term “treatment” includes amelioration of the symptoms of a disease, disorder or condition, or reducing the severity of a disease, disorder or condition. Similarly, “prevention” does not necessarily imply that a subject will never contract a disease, disorder or condition. “Prevention” may be considered as reducing the likelihood of onset of a disease, disorder or condition, or preventing or otherwise reducing the risk of developing a disease, disorder or condition. For example, “prevention” in the context of skin cancers and skin precancers may include decreasing the risk of developing skin cancers or skin precancers.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human.

The immunosuppressant agent that binds to FKBP12 may form a complex with FKBP12 and a further molecule (such as calcineurin or mTOR). Tacrolimus forms a complex with FKBP12 and calcineurin. Sirolimus and everolimus form a complex with FKBP12 and mTOR. The immunosuppressant agent that binds to FKBP12 may be tacrolimus, sirolimus or everolimus, especially tacrolimus. In another embodiment, the immunosuppressant agent that binds to FKBP12 is tacrolimus, everolimus, sirolimus (rapamycin), temsirolimus or zotarolimus; especially tacrolimus or sirolimus; more especially tacrolimus. As the compound of Formula (I) is a tacrolimus antagonist that inhibits tacrolimus through forming a complex with FKBP12, the inventors believe that the compound of Formula (I) would similarly be able to act as antagonists of other FKBP12-binding immunosuppressants such as sirolimus and everolimus.

The subject may be an organ transplant recipient. The only immunosuppressive agent administered to the subject may be an immunosuppressant agent that binds to FKBP12 (especially tacrolimus). The subject may be administered a combination of immunosuppressive agents which includes an immunosuppressant agent that binds to FKBP12 (especially tacrolimus). Exemplary immunosuppressive agents may include calcineurin inhibitors such as cyclosporine A and tacrolimus; antiproliferative agents such as mycophenolate mofetil, mycophenolate sodium and azathioprine; mTOR inhibitors such as sirolirnus; steroids such as prednisone or prednisolone; and/or antibodies such as basiliximab. The transplanted organ may be selected from the group consisting of: liver, kidney, pancreas, heart, lung, trachea, intestine, eye, cornea, face, limb (such as arm, leg, foot and hand), bone and bone marrow. The immunosuppressant agent that binds to FKBP12 may be systemically administered to the subject.

As used herein, “effective amount” refers to the administration of an amount of a compound of Formula (I) or prodrug thereof sufficient to at least partially attain the desired response, or to prevent the occurrence of symptoms of the disease, disorder or condition being treated, or to bring about a halt in the worsening of symptoms or to treat and alleviate or at least reduce the severity of the symptoms. The amount may vary depending on factors such as: the health and physical condition of the individual to whom the compound is administered, the taxonomic group of the individual to whom the compound is administered, the extent of treatment/prevention desired, the formulation of the composition, and the assessment of the medical situation. It is expected that the “effective amount” will fall within a broad range that can be determined through routine trials. An effective amount in relation to a human patient, for example, may lie in the range of about 0.1 ng per cm² of skin to 1 g per cm² of skin per dosage, or in the range of about 1 ng to 100 mg per cm² of skin per dosage, or in the range of about 100 ng to 10 mg per cm² of skin per dosage. Dosage regimes may be adjusted to provide the optimum therapeutic response. For example, several doses may be administered daily, bi-weekly or weekly, or at other suitable time intervals, or the dose may be proportionally reduced as indicated by the circumstances. Decisions on dosage and the like would be within the skill of the medical practitioner or veterinarian responsible for the care of the patient.

In one embodiment, the method of the first aspect prevents or treats skin cancer by reducing the size or volume of a skin cancer or skin precancer, or eradicates or eliminates a skin cancer or skin precancer. In another embodiment, the method of the first aspect prevents or treats skin cancer by preventing the skin cancer or skin precancer from growing, or in the case of a skin precancer from developing into a skin cancer.

In the first aspect, the compound of Formula (I) or prodrug thereof is administered locally to the skin. While it is possible that the compound of Formula (I) (or prodrug thereof) may be administered as a neat chemical, it also may be administered as part of a pharmaceutical composition which includes at least one carrier or excipient. In one embodiment, the compound of Formula (I) or prodrug thereof is administered to the epidermis.

The nature of the pharmaceutical composition and the carrier or excipient will depend on the nature of the disease, disorder or condition and the patient being treated. It is believed that the choice of a particular carrier, excipient or delivery system, and route of administration could be readily determined by a person skilled in the art, and those of skill in the art would be able to prepare suitable formulations. The pharmaceutical composition may include any suitable effective amount of the active agent commensurate with the intended dosage range to be employed.

The pharmaceutical composition may be in the form of a solid, a liquid or a paste; especially a liquid or paste. Exemplary liquids or pastes include solutions, suspensions, syrups, emulsions, colloids, elixirs, creams, gels and foams. The pharmaceutical composition may be a lotion or an ointment. In one embodiment, the ointment has a ratio of oil:water of at least 80:20. In one embodiment, the lotion has a ratio of oil:water of less than 50:50.

The compound of Formula (I) may be administered topically to the skin of the subject. In one embodiment, the compound of Formula (I) or prodrug thereof is administered topically by placing, rubbing or massaging a cream, ointment or salve (or lotion) containing the compound of Formula (I) or prodrug thereof onto the skin. In another embodiment, the compound of Formula (I) or prodrug thereof may be dispersed on or embedded in a bandage, gauze or adhesive (or the like) and placed on the skin. In another embodiment, the compound of Formula (I) or prodrug thereof is administered locally to the skin by intradermal injection, especially into a skin cancer or skin precancer.

The pharmaceutically acceptable carrier(s) or excipient(s) must be acceptable in the sense of being compatible with the other components in the composition and not being deleterious to the patient. The pharniaceutically acceptable carrier or excipient may be either a solid or a liquid. The carrier or excipient may act as a diluent, buffer, stabiliser, isotonicising agent, anti-oxidant, solubilizer, lubricant, suspending agent, binder, preservative or an encapsulating material. Suitable carriers and excipients would be known to a skilled person. With regard to buffers, aqueous compositions may include buffers for maintaining the composition at close to physiological pH or at least within a range of about pH 6.0 to 9.0.

If the pharmaceutical composition is a powder, the active agent (the compound of Formula (I) or prodrug thereof) and a carrier or excipient may both be finely divided powders which are mixed together.

Liquid form preparations may include, for example, water, saline, water-dextrose, water-propylene glycol, petroleum, or oil (including animal, vegetable mineral or synthetic oil) solutions.

Liquid pharmaceutical compositions may be formulated in unit dose form. For example, the compositions may be presented in ampoules, pre-filled syringes, small volume infusion or in multi-dose containers. Such compositions may include a preservative. The compositions may also include formulatory agents such as suspending, stabilising and/or dispersing agents. The composition may also be in powder form for constitution with a suitable vehicle (such as sterile water) before use. Liquid carriers and excipients may include colorants, flavours, stabilizers, buffers, artificial and natural sweeteners, dispersants, thickeners, solubilizing agents, suspending agents and the like.

For local administration to the epidermis the compounds may be formulated as an ointment, cream or lotion, or as a transdermal patch.

The pharmaceutical composition may be in unit dosage form. In such form, the pharmaceutical composition may be prepared as unit doses containing appropriate quantities of the active agent. The unit dosage form may be a packaged preparation, the package containing discrete quantities of preparation.

The compound of Formula (I) (or prodrug thereof) may be administered with a further active agent. For example, the compound of Formula (I) (or prodrug thereof) for administration to the epidermis may be administered with moisturising agents or UV protectants.

In one embodiment, less than 10% (especially less than 5%, 4%, 3%, 2%, 1%, 0.5%, 0.1%, 0.05% or 0.01%) of the compound of Formula (I) (or prodrug thereof) penetrates beyond the dermis after local administration to the skin of the subject. In another embodiment, substantially no (especially no) compound of Formula (I) (or prodrug thereof) penetrates beyond the dermis after local administration to the skin of the subject. In a further embodiment, less than 10% (especially less than 5%, 4%, 3%, 2%, 1%, 0.5%, 0.1%, 0.05% or 0.01%) of the compound of Formula (I) (or prodrug thereof) enters the blood stream after local administration to the skin of the subject. In another embodiment, substantially no (especially no) compound of Formula (I) (or prodrug thereof) enters the blood stream after local administration to the skin of the subject.

In one embodiment, when the subject is an organ transplant recipient, local administration of the compound of Formula (I) (or prodrug thereof) to the skin of the subject may not result in organ transplant rejection.

In another embodiment, the local administration of the compound of Formula (I) or prodrug thereof may not result in systemic effects in the subject.

In one embodiment, administration to the skin of the subject is administration to the surface of the skin of the subject, especially administration to the epidermis of the subject.

In a second aspect, the present invention provides a use of a compound of Formula (I) or a prodrug thereof,

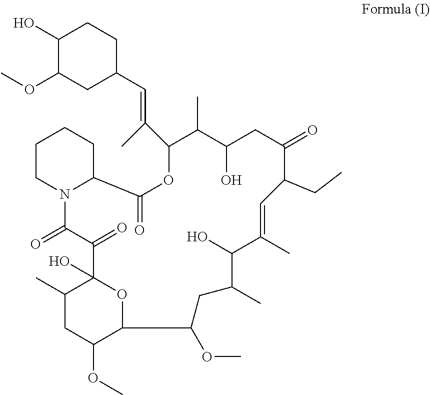 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

in the manufacture of a medicament for preventing or treating skin cancer or skin precancer, wherein the medicament is administered locally to the skin of a subject being administered an immunosuppressant agent that binds to FKBP12.

In a third aspect, the present invention provides a compound of Formula (I) or a prodrug thereof,

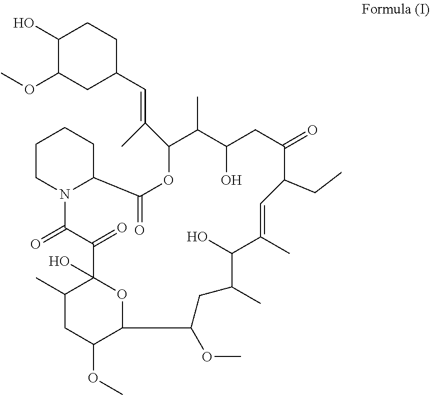 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

for use in preventing or treating skin cancer or skin precancer, wherein the compound of Formula (I) or prodrug thereof is administered locally to the skin of a subject being administered an immunosuppressant agent that binds to FKBP12.

Features of the second and third aspects of the present invention may be as described for the first aspect of the present invention.

In a fourth aspect, the present invention provides a method of preventing or treating a skin condition, disorder or disease associated with administration of an immunosuppressant agent that binds to FKBP12, the method comprising locally administering to the skin of a subject in need thereof an effective amount of a compound of Formula (I) or a prodrug thereof,

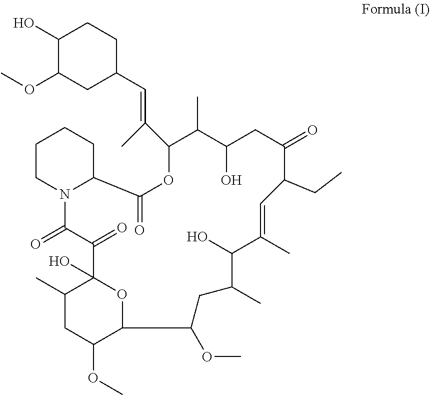 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

wherein the subject is being administered an immunosuppressant agent that binds to FKBP12.

In a fifth aspect, the present invention provides a use of a compound of Formula (I) or a prodrug thereof,

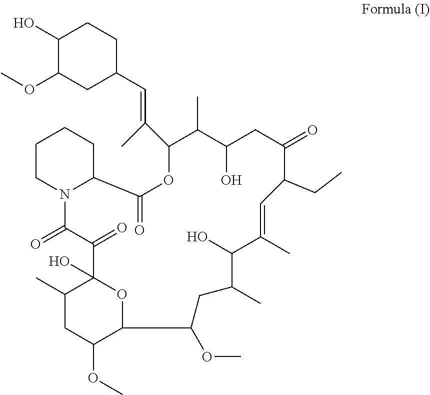 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

in the manufacture of a medicament for the prevention or treatment of a skin condition, disorder or disease associated with administration of an immunosuppressant agent that binds to FKBP12, wherein the medicament is administered locally to the skin of a subject being administered an immunosuppressant agent that binds to FKBP12.

In a sixth aspect, the present invention provides a compound of Formula (I) or a prodrug thereof,

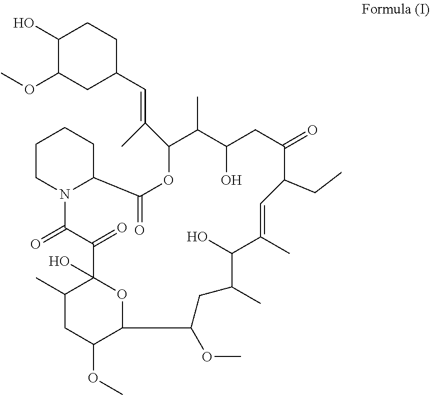 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

for use in the prevention or treatment of a skin condition, disorder or disease associated with administration of an immunosuppressant agent that binds to FKBP12, wherein the compound of Formula (I) or prodrug thereof is administered locally to the skin of a subject being administered an immunosuppressant agent that binds to FKBP12.

Features of the fourth to sixth aspects of the present invention may be as described for the first to third aspects of the present invention.

In one embodiment of the fourth to sixth aspects of the present invention, the immunosuppressant agent that binds to FKBP12 is tacrolimus. In one embodiment of the fourth to sixth aspects of the present invention, the skin condition, disorder or disease associated with administration of an immunosuppressant agent that binds to FKBP12 is skin cancer or skin precancer (as outlined above). In another aspect, the skin condition, disorder or disease associated with administration of an immunosuppressant agent that binds to FKBP12 is a skin condition, disorder or disease which is caused by immune suppression (especially tacrolimus-mediated immune suppression, or sirolimus (rapamycin)-mediated immune suppression). The skin condition, disorder or disease may include a sore (including open sore), lesion, rash, ulcer, wart, inflammation, infection and the like; especially a sore (including open sore), lesion, rash, ulcer, wart, inflammation, and the like.

The subject may have the skin condition, disorder or disease as a result of having a suppressed immune system or other direct effects of the immunosuppressant agent that binds to FKBP12 on the cells in the skin. In one embodiment, the skin condition, disorder or disease may be a hair or nail condition, disorder or disease. The skin condition, disorder or disease may be associated with the epidermis, the dermis, the hypodermis, or a mucous membrane (including oral, nasal, gastrointestinal, penile, vaginal, and conjunctival tissues). The skin condition, disorder or disease may be associated with the hair follicles, or the skin associated with a nail (including the nail bed). The subject may have the skin condition, disorder or disease as a result of having a suppressed immune system or other direct effects of the immunosuppressant agent that binds to FKBP12 on the cells in the skin. Such a skin condition, disorder or disease may include a skin infection (such as a fungal, parasitic, yeast, viral or bacterial infection). Local treatment to the skin (especially topical treatment) with the compound of Formula (I) (or a prodrug thereof) may restore immune function in the skin and result in treatment of the condition, disorder or disease.

Skin conditions, disorders or diseases may be selected from the group consisting of: skin cancer (including malignant skin cancers such as cSCC), skin precancer, fungal infections, parasitic infections, yeast infections, viral infections, bacterial infections, inflammatory skin conditions (including dermatitis, acne, and rosacea), vascular skin conditions (including ulcers and gangrene) and benign skin lesions (including HPV-related warts and actinic keratoses). In one embodiment, the skin condition, disorder or disease may be selected from the group consisting of: skin cancer (including malignant skin cancers such as cSCC), skin precancer (including actinic keratosis (AK), an intraepidermal carcinoma (IEC, such as Bowen's disease) or Kaposi's sarcoma), a fungal infection, a parasitic infection, a yeast infection, a viral infection (including warts (including Molluscum Contagiosum), and Herpes virus (including recalcitrant Herpes virus), a bacterial infection, an inflammatory skin condition (including dermatitis (such as acneiform dermatitis), acne, and rosacea), a vascular skin condition (including ulcers and gangrene), pruritis (itching), folliculitis, onychopathy (including onycholysis, fragile nails or ridged nails), lesions or sores (including an ulcer (including an oral ulcer), a benign skin lesion (including HPV-related warts and actinic keratoses)), poor wound healing (or slow wound healing), a rash (including an exanthem, such as a maculopapular exanthem), oedema (including angioedema), stomatitis, hair loss and hypertrichosis. In one embodiment, the skin condition, disorder or disease is selected from the group consisting of: pruritis, folliculitis, onychopathy, a lesion or sore, poor wound healing, a rash, oedema, stomatitis, hair loss and hypertrichosis. In one embodiment, the skin condition, disorder or disease associated with administration of an immunosuppressant agent that binds to FKBP12 is a skin condition, disorder or disease associated with systemic administration of an immunosuppressant agent that binds to FKBP12.

In one embodiment of the fourth to sixth aspects of the present invention, the compound of Formula (I) may be administered topically to the skin of the subject (or to the skin, nails or hair of the subject), especially to the skin. In one embodiment, the compound of Formula (I) or prodrug thereof is administered topically by placing, rubbing or massaging a cream, ointment or salve containing the compound of Formula (I) or prodrug thereof onto the skin (or onto the skin, nails or hair). In another embodiment, the compound of Formula (I) or prodrug thereof may be dispersed on or embedded in a bandage, gauze or adhesive (or the like) and placed on the skin (or skin or nails). In one embodiment, administration to the skin of the subject is administration to the surface of the skin of the subject, especially administration to the epidermis of the subject. In another embodiment, administration to the skin of the subject is administration to the nails of the subject (and the compound of Formula (I) may pass through the nails to the underlying skin).

In a seventh aspect, the present invention provides a pharmaceutical composition for local administration to the skin which comprises an effective amount of compound of Formula (I) or a prodrug thereof

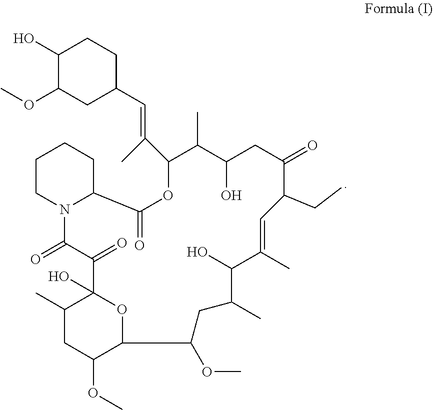 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]

In one embodiment of the seventh aspect, the composition may be for administration to a subject being administered an immunosuppressant agent that binds to FKBP12, especially tacrolimus. The composition may further comprise a pharmaceutically acceptable carrier, diluent and/or excipient. The composition may be for administration to the skin (or nails), especially the epidermis.

Features of the seventh aspect of the present invention may be as described for the first to sixth aspects. The medicament of the second and fifth aspects of the present invention may be a pharmaceutical composition, as described above.

Unless defined otherwise, all technical and scientific terms used herein have the same meaning as would be commonly understood by those of ordinary skill in the art to which this invention belongs.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In the present specification and claims, the word ‘comprising’ and its derivatives including ‘comprises’ and ‘comprise’ include each of the stated integers but does not exclude the inclusion of one or more further integers.

Any of the features described herein can be combined in any combination with any one or more of the other features described herein within the scope of the invention.

### BRIEF DESCRIPTION OF DRAWINGS

Examples of the invention will now be described by way of example with reference to the accompanying Figures, in which:

FIG. 1 is an illustration of tacrolimus (FK506) binding to its cellular target (the protein FKBP12) and calcineurin;

FIG. 2 shows time resolved fluorescence resonance energy transfer (TR-FRET) assay results. FIG. 2A shows the result of a time resolved fluorescence resonance energy transfer (TR-FRET) assay for Tacrolimus (FK506) binding to the FKBP12 enzyme. FIG. 2B shows the result of a time resolved fluorescence resonance energy transfer (TR-FRET) assay for Compound 1 binding potently to the FKBP12 enzyme in the same concentration range as tacrolimus; data is representative of N=2;

FIG. 3 shows the results of mouse T cell proliferation assays. FIG. 3A shows the dose dependent rescue of CD8+ T cell proliferation by Compound 1 (Cmpd 1) in the presence of tacrolimus. FIG. 3B shows that Compound 1 (Cmpd 1) in the absence of tacrolimus had no significant effect on CD8+ T cell proliferation. FIG. 3C shows that Compound 1 (Cpmd 1) in the absence of tacrolimus had no significant effect on CD8+ T cell viability;

FIG. 4 shows the results of a human T cell proliferation assay. FIG. 4A shows the proliferation of human T cells with tacrolimus and Compound 1 (Cmpd 1). FIG. 4B shows the proliferation of human T cells with Compound 1 (Cmpd 1) alone;

FIG. 5 shows the results of a human T cell proliferation assay. FIG. 5A shows the proliferation of human T cells with rapamycin and Compound 1 (Cmpd 1). FIG. 5B shows the proliferation of human T cells with cyclosporine A and Compound 1 (Cmpd 1);

FIG. 6 shows results from a mouse cSCC tumour model. FIG. 6A shows the effect of Compound 1 (Cmpd 1) on tacrolimus dependent-tumour growth compared to the Vehicle control. FIG. 6B shows the effect of Compound 1 (Cmpdl) on activation of tacrolimus-suppressed CD8 T cells from mouse tumours. FIGS. 6C and 6D shows the effect of Compound 1 (Cmpd 1) on interferon gamma or TNF alpha cytokine production by tacrolimus-suppressed CD8 T cells from mouse tumours;

FIG. 7 shows the results from a mouse tumour model. FIG. 7A shows the effect of Compound 1 (Cmpd 1) on tacrolimus dependent-tumour growth after CD8 T cell depletion with a CD8b antibody compared to an isotype control suggesting that the antitumor effect of Compound 1 is via CD8 T cells; and

FIG. 8 shows the results from a mouse spindle cell sarcoma tumour model (Kaposi sarcoma). FIG. 8A shows the effect of Compound 1 (Cmpd 1) on tacrolimus dependent-tumour growth compared to the Vehicle control. FIGS. 8B and 8C shows the effect of Compound 1 (Cmpd 1) on interferon gamma or TNF alpha cytokine production by tacrolimus-suppressed CD8 T cells from mouse tumours.

Preferred features, embodiments and variations of the invention may be discerned from the following Examples which provides sufficient information for those skilled in the art to perform the invention. The following Examples are not to be regarded as limiting the scope of the preceding Summary of the Invention in any way.

### EXAMPLES

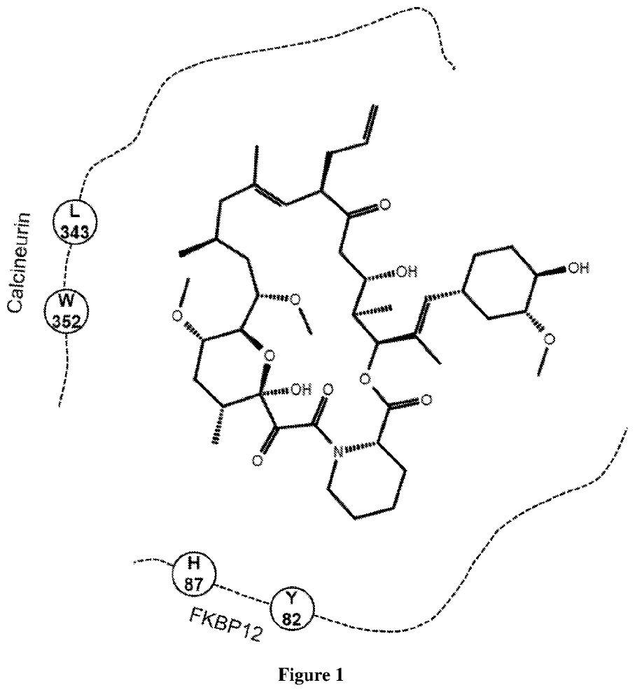

Examples of the present invention will now be described with reference to FIGS. 1 to 8.

### Example 1

### Synthesis of 17-Ethyl-1,14,20-trihydroxy-12-[2′-(4″-hydroxy-3″-methoxycyclohexyl)-1′-methylvinyl]-23,25-dimethoxy-13,19,21,27-tetramethyl-11,28-dioxa-4-azatricyclo-[22.3.1.0^{4,9}]octacos-18-ene-2,3,10,16-tetraone (Compound I)

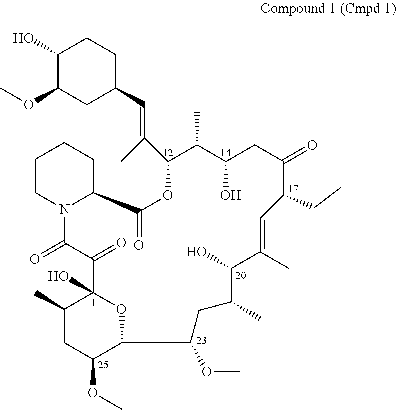 [SMILES: CC[C@@H]1/C=C(\C)[C@@H](O)[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

Reaction was performed as 5 parallel batches and combined for purification. To a stirred solution of 17-ethyl-1,14-dihydroxy-12-[2′-(4″-hydroxy-3″-methoxycyclohexyl)-1′-methylvinyl]-23,25-dimethoxy-13,19,21,27-tetramethyl-11,28-dioxa-4-azatricyclo-[22.3.1.0^{4,9}]octacos-18-ene-2,3,10,16-tetraone (2.0 g, 2.5 mmol) (ascomycin, purchased from Angene Chemical) in a mixture of acetic acid (32 mL) and water (32 mL) was added selenium dioxide (0.42 g, 3.79 mmol) at ambient temperature. The reaction mixture was stirred for 16 h, then a further portion of selenium dioxide (0.42 g, 3.79 mmol) was added, and stirring was continued for 24 h. The reaction mixture was neutralised by the addition of saturated sodium hydrogen carbonate solution, and then extracted with ethyl acetate. The combined organic phase was dried over sodium sulfate and concentrated in vacuo. The residue was purified by silica gel column chromatography (dichloromethane/methanol) then reverse-phase column chromatography (C18 80 g column, PrepChrom C-700 Purification system, 0.1% formic acid in water/acetonitrile) to give 17-ethyl-1,14,20-trihydroxy-12-[2′-(4″-hydroxy-3″-methoxycyclohexyl)-1′-methylvinyl]-23,25-dimethoxy-13,19,21,27-tetramethyl-11,28-dioxa-4-azatricyclo-[22.3.1.0^{4,9}]octacos-18-ene-2,3,10,16-tetraone (3.1 g, 30.4% yield for 5 combined parallel reactions) as a white solid. A portion of the material was further purified by prep-HPLC using the following conditions: Shimadzu UFLC XR, Column: Xterra Prep MS C18 OBD, 19×150 mm, 10 micron, Column temperature: ambient temperature, Mobile Phase A : H2O+0.05% Formic Acid, Mobile Phase B: acetonitrile, Flow rate: 15 mL/min, Mobile phase gradient details: T=0 min (95% A, 5% B); T=1 min (95% A, 5% B); T=9 min (50% A, 50% B); T=13.5 min (50% A, 50% B); T=13.6 min (5% A, 95% B), analysis time 15.5 min.

¹H NMR (400 MHz, Chloroform-d) (selected signals) δ 5.64 (br d, J=9.0 Hz, 1H), 4.11 (br d, J=3.9 Hz, 1H).

LCMS: Rt=2.76 min, [M+Na]+=830, [M+NH₄]+=825, [M+H]+=808. The sample was analysed using the following conditions: Shimadzu LCMS-2020 Nexera UHPLC, Column: Xterra MS-C18, 2.1×50 mm, 3.5 micron, Column temperature: 40° C., Mobile Phase A: H2O+0.05% Formic Acid, Mobile Phase B: acetonitrile, Mobile phase gradient details: T=0 min (95% A, 5% B); T=0.3 min (95% A, 5% B); gradient to T=3 min (5% A, 95% B); end of run at T=4 min (5% A, 95% B), Flow rate: 0.5 mL/min, analysis time 5.5 min.

### Example 2

### TR-FRET Assay (Carried Out by Selcia Discovery, UK)

A 384 well plate time resolved fluorescence resonance energy transfer (TR-FRET) assay was used to determine the effect of inhibitors to compete for tacrolimus (FK506) binding to the FKBP12 enzyme. The FKBP12 enzyme used in the assay is tagged with a polyhistidine sequence. An anti-6xHis antibody, labelled with a fluorescent donor, F(d), binds the tagged enzyme. The enzyme ligand, FK506 is tagged with a fluorescent acceptor, F(a) and binds the enzyme. When the components of the antibody/enzyme/ligand complex are in close proximity, excitation of the F(d) labelled antibody at a particular wavelength A results in F(a) emission at wavelength B due to non-radiative energy transfer. In the presence of a test inhibitor which competes for FK506, the complex is disrupt& and is no longer in close proximity, resulting in emission at wavelength A due to the F(d).

### Assay Protocol for Determination of Binding of Compound 1 to the FKBP12 Enzyme

An 8-point dilution series was performed for Compound 1 and unlabelled tacrolimus (FK506—used as a control) over a concentration range of 0.001 nM to 10 μM. The inhibitors were added to the master mix in the assay plate containing the enzyme/antibody/ligand complex, with a final detergent concentration of 0.005%. The reaction was incubated for 30 min at room temperature and then read on a SpectraMax M5 (Molecular Devices) at wavelengths A (615 nm) and B (665 nm). The ratio between light emission (wavelength B/A) is calculated and the blank subtracted values were plotted against the inhibitor concentration in Logio molar and fitted using one site Kᵢ non-linear regression to determine the Kd of the bound test inhibitor.

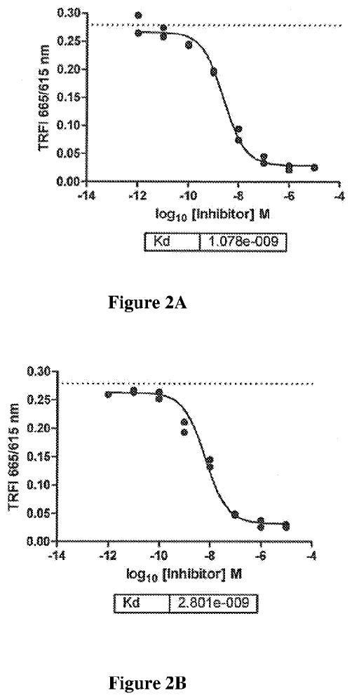

Results: The Kd of the reference compound was determined to be 1.078 nM and the Kd of Compound 1 was 2.801 nM. FIG. 2A shows the result of the assay for tacrolimus (FK506), and FIG. 2B the result for Compound 1.

### Example 3

### Mouse T Cell Proliferation Assay

Compound preparation: Tacrolimus 5 mg/mL stock (FK506—LC Labs) was dissolved in 80% ethanol and Compound 1 10 mM stock was dissolved in DMSO.

### Procedure:

Spleens, inguinal and brachial lymph nodes were harvested from two C57BL/6 mice in sterile conditions and placed in sterile Phosphate Buffered Saline (PBS), then cells were dissociated into a single cell suspension by gently crushing all organs through a 70 μm cell strainer. Cells were washed through the strainer into a 50 mL falcon tube with 5 mL sterile ACK lysis buffer (0.15 M NH₄Cl, 1 mM KHCO₃ pH 7.3). Cells were incubated for 3 min at room temperature to lyse red blood cells. 5 mL sterile PBS was added to the cell suspension and cells were centrifuged at 350 g for 10 min at room temperature. Supernatant was removed and the cell pellet resuspended in 10 mL sterile PBS. Cells were centrifuged again at 350 g for 10 min at room temperature. The supernatant was removed and the cells were resuspended in 1 mL of sterile PBS.

1 μμL of 5 mM CellTrace Violet (Thermo Fisher) was added to the cell suspension and the mixture was swirled to ensure even dispersion of the dye. Cells were incubated for 20 min in the dark at room temperature. 5 mL of pre-warmed complete RPMI medium [(RPMI 1640 (Gibco), 10% heat inactivated FBS (Gibco), 1× Penicillin/Streptomycin/L-Glutamine, 100 μM 2-Mercaptoethanol] was added to the cell suspension and incubated in the dark at room temperature for 5 min. Cells were centrifuged at 350 g for 5 min at room temperature. Supernatant was removed without disrupting the cell pellet. Cells were resuspended in 1 mL complete RPMI medium.

Cells were counted using a haemocytometer and cells were adjusted to 2×10⁶ cells/mL using complete RPMI medium. 500 μL of cell suspension was added to wells in a 48-well plate (Nunc) so the final number of cells per well was 1×10⁶.

A vial of Dynabeads Mouse T Activator CD3/CD28 beads (4×10⁷ beads/mL) was vortexed for 30 sec and 625 μL of beads was aliquoted into a 5 mL polypropylene FACS tube (Sarstedt). 1 mL of complete RPMI medium was added to the beads and mixed by pipetting. The FACS tube was placed in a StemCell Technologies EasySep magnet for 1 minute and supernatant was discarded. The tube was removed from the magnet and washed beads were resuspended in 625 μL of complete RPMI medium. 12.5 μL of bead suspension (5×10⁵ beads) was added into appropriate wells of the 48-well plate with cell suspensions to induce T cell activation.

Tacrolimus or vehicle control was diluted in complete RPMI medium and added to appropriate wells with cell suspension for a final concentration of 0.6 ng/mL. Cells were incubated in a humidified incubator at 37° C. with 5% CO₂ for 1 hour before Compound 1 (or vehicle only) was diluted in complete RPMI medium and added to the appropriate wells at the concentration required. The final volume in all wells was 1 mL. Cells were incubated for 3 days in a humidified incubator at 37° C. with 5% CO₂.

Cells were harvested from each well by resuspending cells with a pipette and transferring to polypropylene FACS tubes. Any remaining cells were washed off wells using an additional 1 mL FACS buffer and transferred to appropriate FACS tubes. Cells were pipetted up and down to remove attached cells from CD3/CD28 beads. FACS tubes with cells were placed in a StemCell Technologies EasySep magnet for 1-2 min to separate magnetic CD3/CD28 beads from solution. Supernatant containing cells were transferred to appropriate polystyrene FACS tubes. 5 mL FACS buffer was added to FACS tubes and cells were centrifuged at 350 g for 5 min at 4° C. Supernatant was removed and cells were resuspended in 5 mL of FACS buffer (2% FBS in PBS) and centrifuged again at 350 g for 5 min at 4° C. Supernatant was removed leaving the cell pellet.

Mouse FC block (Biolegend) was diluted 1:200 in FACS buffer. 50 μL of diluted mouse FC block was added to each tube with cells. Tubes were incubated on ice in the dark for 15 min. A mix of fluorescent antibodies including CD45.2-PE-Cy7, TCRb-FITC and CD8b-APC (all Biolegend) was prepared at a 1:200 dilution in FACS buffer. After the 15 min incubation in FC block 50 μL of diluted antibody solution was added to each tube. Tubes were incubated on ice in the dark for 30 min. 5 mL of FACS buffer was added to each tube and tubes were centrifuged at 350 g for 5 min at 4° C. Supernatant was removed and cell pellet was resuspended in 500 μL of FACS buffer. 5 μL of 7AAD live/dead discrimination dye (Thermo Fisher) was added to each sample 15 min before acquiring samples on an LSR Fortessa X20. Data was analysed using FlowJo software.

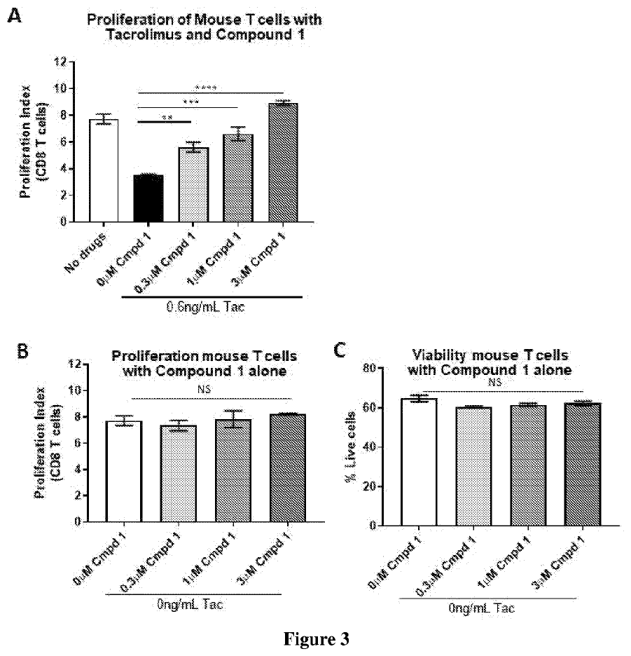

Compound I (Cmpd 1) rescues mouse CD8+ T cell proliferation in vitro in the presence of tacrolimus: Lymphocytes from mouse spleen and lymph nodes were stained with Cell Trace Violet, then pre-incubated with 0.6 ng/mL tacrolimus (Tac) or vehicle control (No tacrolimus used in FIGS. 3B and 3C) for 1 h before Compound 1 was added at concentrations 0, 0.3 μM, 1 μM or 3 μM as indicated. T cells were stimulated with Dynabeads Mouse T Activator CD3/CD28 beads and allowed to proliferate at 37° C. with 5% CO₂ in a humidified incubator for 3 days. Proliferation of CD8+ T cells was assessed by Cell Trace Violet dilution via flow cytometry. Viability was assessed by the 7AAD live/dead discrimination dye via flow cytometry. Proliferation Index on the Y axis was calculated by analysing proliferation peaks of Cell Trace Violet dye in live cells to calculate the average number of cell divisions completed on average by a proliferating cell.

Results depicted in FIG. 3A shows 0.6 ng/mL Tacrolimus inhibited CD8+ T cell proliferation compared to the vehicle control (no drugs), while 0.3 μM, 1 μM and 3 μM Compound 1 dose dependently and significantly rescued CD8+ T cell proliferation in the presence of tacrolimus. Compound 1 in the absence of Tacrolimus had no significant effect on CD8+ T cell proliferation (FIG. 3B) or viability (FIG. 3C). Samples were assayed in triplicate and error bars are SEM. Representative of n=2 experiments. Significance determined by ANOVA.

### Example 4: Human T Cell Proliferation Assay

Compound preparation: Tacrolimus 5 mg/mL stock (FK506—LC Labs) was dissolved in 80% ethanol and Compound 1 (Cmpd 1) 10 mM stock was dissolved in DMSO. Cyclosporine A 1 mg/mL stock (CsA—Sigma) was dissolved in DMSO, Rapamycin 5 mg/mL stock (LC Labs) was dissolved in ethanol.

### Procedure:

Under sterile conditions, a 96 well flat bottom plate with white opaque walls (Perkin Elmer) was coated with 10 μg per well of purified human anti-CD3 antibody (clone OKT-3; Biolegend) in a volume of 50 μL per well in sterile PBS. The plate was covered in parafilm and incubated overnight at 4° C. Just prior to addition of Peripheral Blood Mononuclear Cells (PBMCs), the antibody solution was removed and discarded using a multichannel pipette. Wells were washed by adding 200 μL sterile PBS to each well and incubating for 2 min. PBS wash solution was removed and discarded following incubation. The wash step was repeated once, for a total of two PBS washes.

20 mL of blood from a consenting healthy volunteer was collected into lithium heparin vacuette containers (Greiner Bio-One). Under sterile conditions, blood from each container was pooled into a single tube and mixed thoroughly with 20 mL FACS buffer (2% FBS in 1×PBS). 15 mL Ficoll-Paque (GE Healthcare) was added to two 50 mL Falcon tubes, before 20 mL of whole blood/FACS buffer mixture was carefully and slowly dispensed on top of the Ficoll layer. Tubes were centrifuged at 800 g for 20 min at room temperature with the brake off. After centrifugation, the resulting PBMC layer between the plasma and Ficoll layers was carefully removed and transferred to a new 50 mL Falcon tube. Tubes were topped up to 45 mL with FACS buffer, then centrifuged at 500 g for 15 min. Supernatant was carefully removed without disturbing cell pellet, then PBMCs from both tubes were pooled. 30 mL of pre-warmed complete RPMI media [RPMI 1640 (Gibco), 10% heat inactivated FBS (Gibco), 1× Penicillin/Streptomycin/L-Glutamine (Gibco), 100 μM 2-Mercaptoethanol (Sigma)] was added to PBMCs, before centrifugation at 500 g for 15 min at room temperature. Supernatant was removed carefully without disturbing cell pellet. PBMCs were resuspended in 1 mL complete RPMI media.

PBMCs were counted manually using a haemocytometer and cell concentration was adjusted to 1.5×10⁶ cells/mL with complete RPMI media. 50 μL of cell suspension was added to appropriate wells of the 96 well flat bottom plate with white opaque walls pre-coated in human anti-CD3. The final number of cells per well was 7.5×10⁴.

Tacrolimus, Cyclosporine A, Rapamycin (sirolimus) or vehicle control was diluted in complete RPMI medium and added to appropriate wells with PBMC suspension at the concentration required. Cells were incubated in a humidified incubator at 37° C. with 5% CO₂ for 1 hour before Compound 1 (or vehicle only) was diluted in complete RPMI medium and added to the appropriate wells at the concentration required. The final volume in all wells was 100 μL. Cells were incubated for 5 days in a humidified incubator at 37° C. with 5% CO₂.

CellTiter-Glo Luminescent Cell Viability Assay (Promega) was performed to determine the level of metabolically active cells per well based on ATP quantification. CellTiter-Glo Buffer and lyophilised CellTiter-Glo Substrate were equilibrated to room temperature. CellTiter-Glo Substrate was reconstituted with 10 mL CellTiter-Glo Buffer and gently vortexed for 1 minute to create the CellTiter-Glo Reagent. The 96 well plate containing PBMCs was equilibrated to room temperature for 30 min. Once at room temperature, 100 μL of CellTiter-Glo Reagent was dispensed using a multichannel pipette into each well containing 100 μL cells and media. The plate was mixed for 2 min at room temperature on an orbital shaker to induce cell lysis. The plate was then incubated for 10 min at room temperature in the dark to stabilize the luminescence signal. Luminescence signal was recorded using a CLARIOstar Plus plate reader (BMG Labtech).

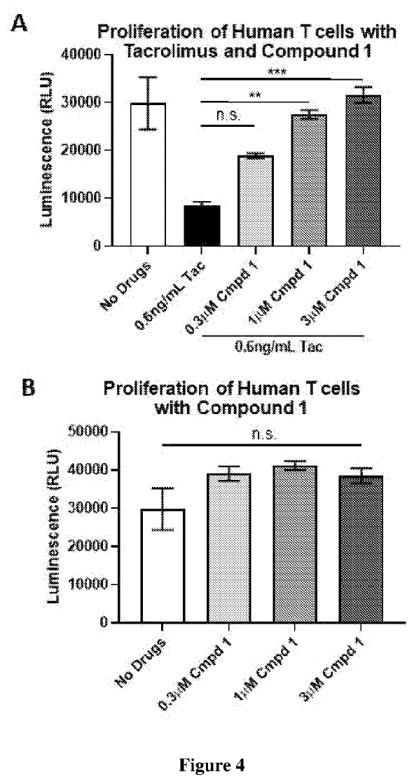

Compound I (Cmpd 1) rescues human T cell proliferation in vitro in the presence of tacrolimus: PBMCs isolated from human blood were pre-incubated with 0.6 ng/mL tacrolimus (Tac) or vehicle control (no tacrolimus used in FIG. 4B) for 1 h before Compound 1 (Cmpd 1) was added at concentrations 0, 0.3 μM, 1 μM or 3 μM as indicated. PBMCs were stimulated with human anti-CD3 antibody and T cells were allowed to proliferate at 37° C. with 5% CO₂ in a humidified incubator for 5 days. Proliferation/viability was assessed via a CellTiter-Glo Luminescent Cell Viability Assay and luminescence was read using a CLARIOstar Plus plate reader.

Results depicted in FIG. 4A shows 0.6 ng/mL Tacrolimus (Tac) inhibited T cell proliferation compared to the vehicle control (no drugs), while 1 μM and 3 82 M Compound 1 dose dependently and significantly rescued T cell proliferation in the presence of tacrolimus. FIG. 4B shows that Compound 1 alone did not significantly alter T cell proliferation/viability. Samples were assayed in triplicate and error bars are SEM. Significance determined by ANOVA. Representative of n=2 experiments.

Compound 1 (Cmpd 1) Rescues Human T Cell Proliferation In Vitro in the Presence of FKBP12-binding Rapamycin, but Not Cyclophilin-Binding Cyclosporine A:

PBMCs isolated from human blood were pre-incubated with 1 ng/mL rapamycin (Rapa), 50 ng/mL cyclosporine A (CsA) or vehicle control for 1 h before Compound 1 (Cmpd 1) was added at concentrations 0, 0.3 μM, 1 μM or 3 μM as indicated. PBMCs were stimulated with human anti-CD3 antibody and T cells were allowed to proliferate at 37° C. with 5% CO₂ in a humidified incubator for 5 days. Proliferation/viability was assessed via a CellTiter-Glo Luminescent Cell Viability Assay and luminescence was read using a CLARIOstar Plus plate reader.

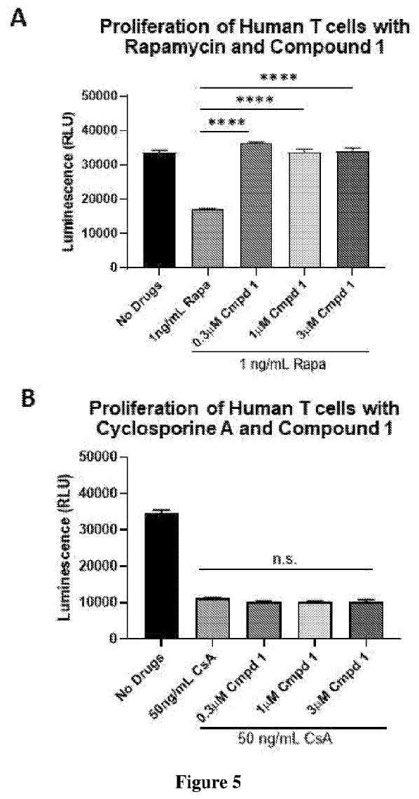

Results depicted in FIG. 5A shows 1 ng/mL rapamycin (Rapa) inhibited T cell proliferation compared to the vehicle control (no drugs), while 0.3 μM, 1 μM and 3 μM Compound 1 significantly rescued T cell proliferation in the presence of rapamycin. In FIG. 5B 50 ng/mL cyclosporine A (CsA) inhibited T cell proliferation compared to the vehicle control (no drugs), and 0.3 μM, 1 μM and 3 μM Compound 1 were unable to rescue T cell proliferation in the presence of cyclosporine A as expected. Samples were assayed in triplicate and error bars are SEM. Significance determined by ANOVA.

### Example 5

### Mouse Tumour Model

Mice: All animal procedures were approved by the University of Queensland Animal Ethics Committee (approval no UQDI/512/17). K14HPV38E6/E7 mice, which express the E6 and E7 genes of Human Papillomavirus (HPV) type 38 under the control of the Keratin 14 promoter (Viarisio et al., “E6 and E7 from Beta HPV38 Cooperate with Ultraviolet Light in the Development of Actinic Keratosis-like Lesions and Squamous Cell Carcinoma in Mice.” PLoS Pathog. 2011 e1002125) were bred and maintained locally at the Translational Research Institute Biological Research Facility (Brisbane, Australia). All mice used were between 12 and 20 weeks and were housed under specific pathogen-free conditions.

Tacrolimus diet: All customised mice diet was manufactured by Specialty Feeds (Perth, Wash.). Briefly, tacrolimus (MedChemExpress) was mixed with caster sugar and then incorporated into standard mouse diet. 1.5 g of tacrolimus was mixed with 100 g of caster sugar and 9.9 kg of standard mouse diet to result in tacrolimus-diet (150 ppm). Food colouring was added to distinguish the drug. During the manufacturing process the pellets were air-dried overnight rather than dried in an oven in order to minimise the amount of heat applied. The final product was sealed in airtight bags and stored at 4° C. protected from light to ensure minimal degradation. Pellet volumes in feed hoppers were kept to a minimum and restocked every 3-4 days for the duration of the experiments.

Compound 1 preparation: Compound 1 (Cmpd 1) was prepared at the beginning of each dosing week as a 1 or 2 mg/mL solution as required in 4% ethanol/0.2% Tween-80/PBS. Compound 1 solution was stored at 4° C. for up to one week.

Mouse back cSCC tumour model: K14-HPV38-E6/E7 mice were randomised into groups based on body weight and age and were fed tacrolimus (150 ppm in the diet) for 7 days prior to tumour cell injection and throughout the study. HPV38-E6/E7 cells (cSCC cell line derived from UV-induced tumours from the mouse strain above) were cultured and passaged for 1 week prior to injection in complete F-12 media [3:1 v/v F-12 (Gibco) and DMEM high glucose (Gibco) medias supplemented with 5% heat inactivated FBS (Gibco), 0.4 μg/mL Hydrocortisone (Sigma), 5 μg/mL Insulin (Sigma), 8.4 ng/mL Cholera Toxin (Sigma), 10 ng/mL Human rEGF (Invitrogen), 24 μg/mL Adenine (Sigma), 1× Penicillin/Streptomycin/Glutamine (Gibco)]. Cells were washed twice in PBS prior to injection. Mice were injected with 1×10⁶ HPV38-E6/E7 SCC cells in PBS subcutaneously into the center of the shaved lower back in a 100 μL volume using a 30G syringe. Tumour size was monitored 3× weekly throughout the study using digital callipers. Once tumours reached approximately 0.05-0.1 cm³ mice were treated twice daily (as required) with intratumoural injections (40 μL) with Compound 1 or vehicle for up to 4 weeks or until euthanasia criteria were met. Mice were euthanased once tumours reached 1 cm³.

Mouse back 5117-RE tumour model: Method as per the above section (Mouse back cSCC tumour model), with the following changes; BALBc mice were used, and 5117-RE cells were cultured and passaged for 1 week prior to tumour cell injection in RPMI media (Gibco) supplemented with 10% heat inactivated FBS (Gibco) and 1× Penicillin/Streptomycin/Glutamine (Gibco).

CD8 T cell depletion: CD8b depleting antibody (BioXCell; clone 53-5.8) or isotype control antibody (BioXCell; clone HRPN) were administered by intraperitoneal injection on days 8, 15, and 22 post SCC challenge. 250 μg per mouse in 200 μL PBS was administered on days 8 and 15. 100 μg per mouse in 200 μL PBS was administered on day 22. Mice were bled on day 10 post cSCC challenge to check depletion efficiency via FACS.

Dissociation of tumour cells for FACS analysis: To release cells from tumours, harvested tissue was cut into small fragments and digested for 60 min at 37° C. in RPMI media containing 2% FBS, 3 mg/mL collagenase D and 5 ug/mL DNase I. Tissues were then gently pressed through a 70 μm cell strainer to create a single-cell suspension. Cells from each tumour were resuspended into 300 μL of RPMI media with 10% FCS, 1× Penicillin-Streptomycin-Glutamine (Gibco), 100 μM 2-Mercaptoethanol (Sigma), before staining with appropriate antibodies as described below.

Ex vivo stimulation and staining protocol Ji cytokine detection: 100 μL of each suspension of tumour dissociated cells was incubated in 96-well cell culture plates coated with CD3 antibody (clone 145-2C11—Biolegend) along with soluble CD28 antibody (2.5 μg/ml—clone 37.51, Biolegend) at 37° C. for 30 min. As a control (no stimulation), 100 μL of each suspension of tumour dissociated cells was also incubated an uncoated 96-well cell culture plate without soluble CD28 antibody and incubated at 37° C. for 30 min. After 30 min of incubation, 5 μg/mL Brefeldin A was added to all wells, and cells were incubated at 37° C. for a further 3.5 h. After a 4 h total incubation, cells were resuspended in FACS buffer with 5 μg/mL Brefeldin A (eBioscience)+Fc block (Purified Rat Anti-Mouse CD16/CD32: isotype Rat IgG2a, clone: 93, Biolegend) for 20 min on ice to block non-specific antibody staining. Monoclonal antibodies for surface staining (CD45.1-PE-Dazzle, TCRh-FITC, CD8a-PE-Cy7, CD4-Ax700) were subsequently added and incubated on ice for 30-40 min in concert with Live/Dead Aqua. Stain (Biolegend) to elucidate live cell populations. Cells were then resuspended in fixation buffer (eBioscience), and incubated in the dark at room temperature for 20 min. Cells were washed and resuspended in 1× Permeabilisation buffer (eBioscience)+intracellular antibodies including interferon gamma (IFNg-APC clone XMG1.2—eBioscience) and TNF alpha (TNFa-BV785 clone MP6-XT22—Biolegend). Cells were incubated in the dark at room temperature for 20 min.

CD59 staining protocol: 100 μL of tumour dissociated cells was resuspended in FACS buffer and incubated with Fe-block (Purified Rat Anti-Mouse CD16/CD32: isotype Rat IgG2a, clone: 93, Biolegend) for 20 min on ice to block non-specific antibody staining. Monoclonal antibodies for surface staining (CD45.1-PE-Dazzle. TCRb-FITC, CD8a-PE-Cy7, CD4-Ax700, CD69-APC; Biolegend) were subsequently added and incubated on ice for 30 min in concert with Live/Dead Aqua Stain (Biolegend) to elucidate live cell populations. Cells were then resuspended in fixation buffer and incubated at morn temperature for 20 min.

FACS analysis: Stained tumour dissociated cells were then washed twice, resuspended in FACS buffer and Flow cytometric analysis was performed using LSR Fortessa X20 (BD Biosciences) flow cytometers with FACSDiva. software (Becton Dickinson, Sparks, Md., USA). Data were exported and analyzed using FlowJo software (I'reestar Inc., Ashland, Oreg., USA).

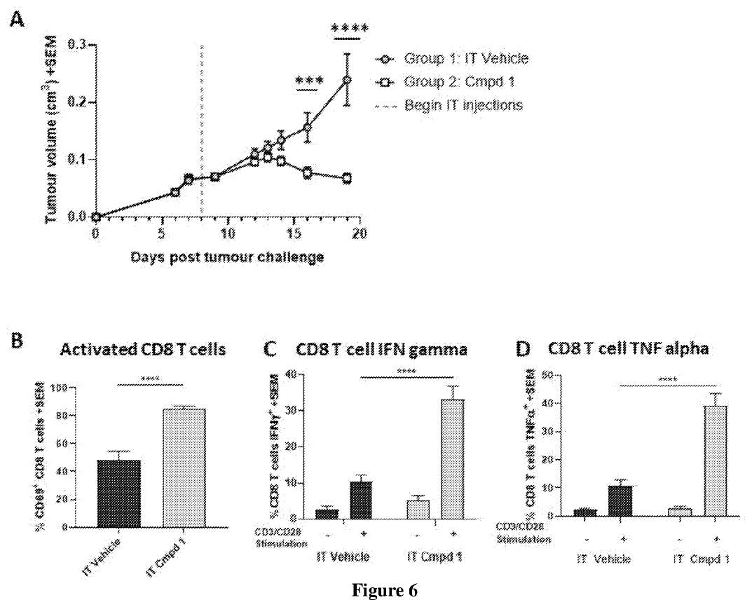

Intra-tumoural injection of Compound 1 (Cmpd 1) significantly reduced tacrolimus-dependent cutaneous Squamous Cell Carcinoma (cSCC) tumour growth: K14-HPV38-E6/E7 mice were fed tacrolimus (150 ppm in the diet) throughout the experiment, beginning 7 days prior to tumour cell injection. Mice were injected sub-cutaneously with 1×10⁶ HPV38-E6/E7 SCC cells into the lower back and tumour size was monitored 3 times a week throughout the experiment. When tumours reached approximately 0.06 cm³ (Day 8), BID intra-tumoural (TT) injections were performed with 40 μL of 2 mg/mL Compound 1 or vehicle control for 12 days. Results depicted in FIG. 6A shows that Compound 1 significantly reduced tacrolimus dependent-tumour growth compared to the vehicle control after 8 days treatment, with regression from peak tumour volume observed. Error bars represent SEM (n=10-11), statistical significance determined by 2-way ANOVA.

Intra-tumoural injection of Compound 1 (Cmpd 1) significantly increased cSCC tumour-infiltrating CD8 T cell activation and intracellular interferon gamma and TNF alpha: After 12 days BID IT injections of 2 mg/mL Compound 1 or vehicle control, 10 mice per treatment group were euthanised, tumours harvested, dissociated into single cells and stained for fluorescence activated cell sorting (FACS) analysis. Results depicted in FIG. 6B shows that Compound 1 significantly increased the percentage of CD69⁺ (activated) CD8 T cells isolated from the tumour. FIG. 6C and 6D shows that Compound 1 significantly increased the percentage of IFN gamma⁺ and TNF alpha⁺ CD8 T cells isolated from the tumour after 12 days treatment (after ex vivo stimulation with CD3/CD28). Error bars represent SEM (n=10), statistical significance determined by t-test or 2-way ANOVA as appropriate.

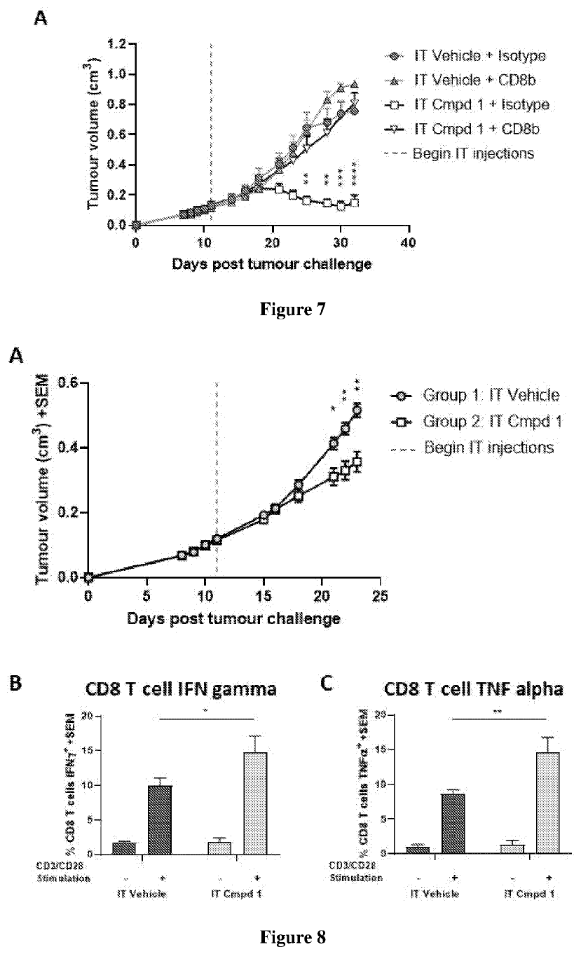

Depletion of CD8 T cells prevents Compound I mediated regression of tacrolimus-dependant cSCC tumours: K14-HPV38-E6/E7 mice were fed tacrolimus (150 ppm in the diet) throughout the experiment, beginning 7 days prior to tumour cell injection. Mice were injected sub-cutaneously with 1×10⁶ HPV38-E6/E7 SCC cells into the lower back and tumour size was monitored 3 times a week throughout the experiment. CD8 T cells were depleted on day 8 by the intraperitoneal injection of CD8b-depleting antibody (CD8b), and again on day 15 and 22 post SCC challenge. When tumours reached approximately 0.1 cm³ (Day 11), BID intra-tumoural (IT) injections were performed with 40 μL of 2 mg/mL Compound 1 or vehicle control for 3 weeks or until euthanasia criteria (tumour size 1 cm³—ethical limit) was reached. Results depicted in FIG. 7 shows that without CD8 depletion (isotype antibody control), Compound 1 significantly reduced tacrolimus dependent-tumour growth compared to the vehicle control as expected, with regression from peak tumour volume observed. After CD8 depletion (CD8b), Compound 1 (Cmpd 1) is no longer able cause tumour regression, with Compound 1+CD8b being significantly different to Compound 1+Isotype antibody control after 14 days treatment. Error bars represent SEM (n=12-16), statistical significance determined by 2-way ANOVA.

Intra-tumoural injection of Compound I (Cmpd 1) significantly reduced tacrolimus-dependent spindle cell sarcoma (5117-RE) tumour growth: BALBc mice were fed tacrolimus (150 ppm in the diet) throughout the experiment, beginning 7 days prior to tumour cell injection. Mice were injected sub-cutaneously with 1×10⁶ 5117-RE (spindle cell sarcoma) cells into the lower back and tumour size was monitored 3 times a week throughout the experiment. This cell line can be considered a model of Kaposi's sarcoma (KS), as KS is generally regarded to be a tumour of spindle cell lineage origin (Duman, Nephrology Dialysis Transplantation, 2002 https://doi.org/10.1093/ndt/17.5.892). When tumours reached approximately 0.1 cm³ (Day 11), BID intra-tumoural (IT) injections were performed with 40 μL of 2 mg/mL Compound 1 or vehicle control for 13 days. Results depicted in FIG. 8A shows that Compound 1 significantly reduced tacrolimus dependent-tumour growth compared to the vehicle control after 10 days treatment. Error bars represent SEM (n=19-20), statistical significance determined by 2-way ANOVA.

Intra-tumoural injection of Compound 1 (Cmpd 1) significantly increased 5117-RE tumour-infiltrating CD8 T cells producing cytokines TNF alpha and interferon gamma: After 13 days BID IT injections with Compound 1 or vehicle control, 10 mice per treatment group were euthanised, 5117-RE tumours harvested, dissociated into single cells and stained for fluorescence activated cell sorting (FACS) analysis. Results depicted in FIG. 8B shows that Compound 1 significantly increased the percentage of IFN gamma⁺ CD8 T cells isolated from the tumour after 13 days treatment (after ex vivo stimulation with CD3/CD28). FIG. 8C shows that Compound 1 significantly increased the percentage of TNF alpha⁺ CD8 T cells isolated from the tumour after 13 days treatment. Error bars represent SEM (n=10), statistical significance determined by 2-way ANOVA.

Intra-Tumoural Injection of Compound 1 (Cmpd 1) and cSCC Tumour Pharmacokinetics Indicates High Levels of Compound 1 in Tumours:

In another study of the mouse back tumour model, the concentration of Compound 1 and tacrolimus in SCC tumours was assessed at 1, 6, and 18 h post final dose. Table 1 below tabulates data demonstrating that average tumour concentrations of Compound 1 were more than 2000-fold that of tacrolimus over 18 h.

Tumour processing: The whole tumour was removed from the mouse. The weight of the tumour was recorded and tumour was placed into an appropriate cryovial on dry ice then transferred to a −80° C. freezer. 3 tumours per time point were assessed.

Sample processing: A LC/MS/MS based bioanalytical method was developed for the simultaneous detection and quantification of Compound 1 and tacrolimus in mouse tumours. Calibration standards and quality control samples were prepared by adding 2.5 μL of stock solutions of test compound of different concentrations into 25 μL of naive mouse blood or skin homogenates. Control samples were prepared by spiking 2.5 μL of water or acetonitrile into 25 μL of naïve mouse blood or skin homogenates. Tumour samples were homogenised in 1 mL of PBS then transferred into polypropylene Eppendorf tubes. 100 μL of 0.1 M zinc sulfate was added into the tubes, vortexed for 10 sec, and 250 μL of HPLC-grade acetonitrile containing internal standard (pimecrolimus) was added, vortexed for 2 min, and centrifuged for 3 min at 800×g. 20-40 μL of the supernatant was analysed by LCMS/MS. Instrument: Acquity UPLC, Waters. Column: Acquity BEH C18 100×2.1 mm, 1.7 micron; Mobile phase A: methanol; Mobile phase B: 5 mM ammonium acetate with 0.1% formic acid; Mobile phase gradient details: T=0 min (10% A, 90% B); T=0.01 min (10% A, 90% B); gradient to T=1.5 min (95% A, 5% B); T=3.2 min (95% A, 5% B). Flow rate: 0.3 mL/min, run time: 4.5 min; Ionisation mode: Electrospray ionisation (positive).

TABLE 1
Concentration of Compound 1 and Tacrolimus in Tumour

| Time | Average Concentration | Average Concentration |
|---|---|---|
| (h) | Compound 1 in tumour (ng/g) | tacrolimus in tumour (ng/g) |
| 1 | 164606 | 33 |
| 6 | 148276 | 73 |
| 18 | 168496 | 52 |

### Example 6: Topical Application of Compound

The single dose pharmacokinetics of Compound 1 (Cmpd 1) was assessed by topical administration of the compound as a formulation in propylene glycol (3% concentration) applied once per day (QD) to one ear only of C57BL/6 female mice, 8 weeks of age (N=2 or 3 per timepoint; samples taken 1, 6 and 24 h post administration of Compound 1).

After a single topical dose of Compound 1 to the ear (10 μL of 3% solution in propylene glycol), measured concentrations of Compound 1 in mouse ears are between 22.8 to 30.7 μg/g over 24 hours. Blood concentrations of Compound 1 were all below the limit of quantitation (LOQ) for all samples (<3.5 ng/mL).

Table 2 below provides tabulated data showing the average concentration of Compound 1 quantified in mouse ears following a single topical administration of a 3% solution of Compound 1 in propylene glycol. The average concentration of Compound 1 was between 22.8 to 30.7 μg/mL over 24 h, with no compound detected in mouse blood down to the limit of quantification (LOQ) (3.5 ng/mL).

TABLE 2
Compound 1 quantified in mouse ears

| Time | Concentration | Average concentration |
|---|---|---|
| (h) | in blood (ng/mL) | in ear (ng/g) |
| 1 | BLQ | 28961 |
| 6 | BLQ | 30714 |
| 24 | BLQ | 22847 |

Limit of Quantitation (LOQ): 3.5 ng/mL;
BLQ: Below Limit of Quantitation in blood

### Methods:

Compound Application: Compound 1 (10 μL of a 3% solution in propylene glycol) was applied to mouse ears using a silicone brush. The brush was cleaned with distilled water and ethanol between applications. The vehicle solution was 100% propylene glycol.

Blood processing: Cryovials were prepared containing 10 μL 0.5 M EDTA and labelled appropriately. Cardiac bleed was performed on mice and blood transferred to an Eppendorf tube. Blood (110 μL) was transferred immediately to a cryovial containing EDTA and well mixed to prevent clotting. The cryovial was placed on dry ice and transferred to a −80° C. freezer.

Ear processing: Before taking ears for pharmacokinetics the skin was cleaned with distilled water and dried with cotton balls/swab before doing 2 tape strips (one piece of tape per strip). The whole ear was removed from the mouse. The weight of the whole ear was recorded and placed into an appropriate cryovial which was placed on dry ice then transferred to a −80° C. freezer.

Sample processing: A LC/MS/MS based bioanalytical method was developed for the detection and quantification of Compound 1 in mouse blood and ears. Calibration standards and quality control samples were prepared by adding 2.5 μL of stock solutions of test compound of different concentrations into 25 μL of naïve mouse blood or ear homogenates. Control samples were prepared by spiking 2.5 μL, of water or acetonitrile into 25 μL of naïve mouse blood or ear homogenates. The blood or ear samples were transferred into polypropylene Eppendorf tubes. 100 μL of 0.1 M zinc sulfate was added into the tubes, vortexed for 10 sec, and 250 μL, of HPLC-grade acetonitrile containing internal standard (pimecrolimus) was added, vortexed for 2 min, and centrifuged for 3 min at 800×g. 20-40 μL of the supernatant was analysed by LCMS/MS, using the same instrument, column, Mobile phase A, Mobile phase B, gradient and other parameters as outlined above for the tumour sample processing.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

### CITATIONS

Aldabagh, Bishr, et al. 2013. Dermatologic Surgery: Official Publication for American Society for Dermatologic Surgery 39 (1 Pt 1): 1-23. https://doi.org/10.1111/j.1524-4725.2012.02558.x.
Azzimonti, B., et al. 2015. The British Journal of Dermatology 172 (1): 64-73. https://doi.org/10.1111/bjd.13172.
Badalamenti, Giuseppe, et al. 2019. Cellular Immunology 343 (September): 103753. https://doi.org/10.1016/j.cellimm.2018.01.013.
Baek, Kwan-Hyuck, et al. 2009. Nature 459 (7250): 1126-30. https://doi.org/10.1038/nature08062.
Barnes, Tristan A., and Eitan Amir. 2017. British Journal of Cancer 117 (4): 451-60. https://doi.org/10.1038/bjc.2017.220.
Berman, Brian, and Clay J. Cockerell. 2013. Journal of the American Academy of Dermatology 68 (1 Suppl 1): S10-19. https://doi.org/10.1016/j.jaad.2012.09.053.
Binnewies, Mikhail, et al. 2018. Nature Medicine 24 (5): 541-50. https://doi.org/10.1038/s41591-018-0014-x.
Black, A. P., et al. 2005. The British Journal of Dermatology 153 (5): 987-91. https://doi.org/10.1111/j.1365-2133.2005.06878.x.
Bottomley, Matthew J., et al. 2016. Journal of the American Society of Nephrology: JASN 27 (5): 1505-15. https://doi.org/10.1681/ASN.2015030250.
Bottomley, Matthew J., et al. 2019. International Journal of Molecular Sciences 20 (8). https://doi.org/10.3390/ijms20082009.
Brun, Miranda, et al. 2013. The Journal of Biological Chemistry 288 (33): 24104-15. https://doi.org/10.1074/jbc.M113.455832.
Buchholz, Malte, et al. 2006. The EMBO Journal 25 (15): 3714-24. https://doi.org/10.1038/sj.emboj.7601246.
Calvo-Turrubiartes, M., et al. 2009. Transplant Immunology 21 (1): 43-49. https://doi.org/10.1016/j.trim.2009.02.002.
Canning, Matthew T., et al. 2006. Journal of Molecular Histology 37 (5-7): 285-91. https://doi.org/10.1007/s10735-006-9034-9.
Carroll, Robert P., et al. 2010. Journal of the American Society of Nephrology: JASN 21 (4): 713-22. https://doi.org/10.1681/ASN.2009060669.
Champiat, Stéphane, et al. 2017. Clinical Cancer Research 23 (8): 1920-28. https://doi.org/10.1158/1078-0432.CCR-16-1741.
Cheung, C. Y., et al. 2012. American Journal of Transplantation 12 (11): 3039-46. https://doi.org/10.1111/j.1600-6143.2012.04209.x.
Chung, Byung Ha, et al. 2014. Transplant Immunology 30 (4): 159-67. https://doi.org/10.1016/j.trim.2014.03.003.
Collett, D., et al. 2010. American Journal of Transplantation 10 (8): 1889-96. https://doi.org/10.1111/j.1600-6143.2010.03181.x.
Crispin, José C, and George C Tsokos. 2020. F1000Research 9 (February): 80. https://doi.org/10.12688/f1000research.21150.1.
Daniel, Dylan, et al. 2003. Journal of Experimental Medicine 197 (8): 1017-28. https://doi.org/10.1084/jem.20021047.
Duncan, J. I. 1994. The Journal of Investigative Dermatology 102 (1): 84-88. https://doi.org/10.1111/1523-1747.ep12371737.
Ekström, Magnus, et al. 2017. International Journal of Chronic Obstructive Pulmonary Disease 12 (October): 2841-47. https://doi.org/10.2147/COPD.S147065.
Engels, Eric A. 2019. American Journal of Transplantation 19 (12): 3223-32. https://doi.org/10.1111/ajt.15495.
Engels, Eric A., et al. 2011. Jama 306 (17): 1891-1901. https://doi.org/10.1001/jama.2011.1592.
Euvrard, Sylvie, et al. 2003. New England Journal of Medicine 348 (17): 1681-91. https://doi.org/10.1056/NEJMra022137.
Feldmeyer, Laurence, et al. 2016. Experimental Dermatology 25 (3): 245-47. https://doi.org/10.1111/exd.12878.
Flohil, Sophie C., et al. 2013. The Journal of Investigative Dermatology 133 (8): 1971-78. https://doi.org/10.1038/jid.2013.134.
Freeman, Andrew, et al. 2014. PloS One 9 (10): el10928. https://doi.org/10.1371/journal.pone.0110928.
Gachet, S., et al. 2013. Leukemia 27 (12): 2289-2300. https://doi.org/10.1038/leu.2013.156.
Glynne, Richard, et al. 2000. Nature 403 (6770): 672-76. https://doi.org/10.1038/35001102.
Gnjatic, Sacha, et al. 2017. Journal for Immunotherapy of Cancer 5 (May). https://doi.org/10.1186/s40425-017-0243-4.
Goldstein, Jill, et al. 2015. Molecular Biology of the Cell 26 (20): 3606-14. https://doi.org/10.1091/mbc.E15-05-0282.
Hanna, Edith, et al. 2016. International Journal of Dermatology 55 (8): 831-44. https://doi.org/10.1111/ijd.13235.
Harwood, C. A., et al. 2017. The British Journal of Dermatology 177 (5): 1217-24. https://doi.org/10.1111/bjd.15956.
Heppt, M. V., et al. 2019. The British Journal of Dermatology 180 (1): 43-50. https://doi.org/10.1111/bjd.17148.
Hock, Barry D., et al. 2012. Nephrology, Dialysis, Transplantation: Official Publication of the European Dialysis and Transplant Association—European Renal Association 27 (1): 402-10. https://doi.org/10.1093/ndt/gfr264.
Horsley, Valerie, et al. 2008. Cell 132 (2): 299-310. https://doi.org/10.1016/j.cell.2007.11.047.
Huang, C.-C., et al. 2008. Oncogene 27 (17): 2422-29. https://doi.org/10.1038/sj.onc.1210888.
Iannacone, Michelle R., et al. 2016. Journal of Investigative Dermatology 136 (7): 1382-86. https://doi.org/10.1016/j.jid.2016.02.804.
Ishdorj, Ganchimeg, et al. 2019. Leukemia & Lymphoma 60 (13): 3204-13. https://doi.org/10.1080110428194.2019.1620941.
Jauliac, Sebastien, et al. 2002. Nature Cell Biology 4 (7): 540-44. https://doi.org/10.1038/ncb816.
Jiang, Hong, et al. 1993. Carcinogenesis 14 (1): 67-71. https://doi.org/10.1093/carcin/14.1.67.
Kamada, Takahiro, et al. 2019. Proceedings of the National Academy of Sciences 116 (20): 9999-10008. https://doi.org/10.1073/pnas.1822001116.
Kambayashi, Yumi, et al. 2013. Acta Dermato-Venereologica 93 (6): 663-68. https://doi.org/10.2340/00015555-1597.
Kaplan, A., et al. 1995. Journal of Dermatological Science 10 (2): 130-38. https://doi.org/10.1016/0923-1811(95)00395-9.
Karashima, Tadashi, et al. 1996. Journal of Dermatological Science 12 (3): 246-54. https://doi.org/10.1016/0923-1811(95)00480-7.
Kawahara, Takashi, et al. 2015. Oncotarget 6 (3): 1582-93.
Kim, Hye-Jung, and Harvey Cantor. 2014. Cancer Immunology Research 2 (2): 91-98. https://doi.org/10.1158/2326-6066.CIR-13-0216.
Kogina, Kazue, et al. 2009. Molecules and Cells 28 (2): 125-30. https://doi.org/10.1007/s10059-009-0114-z.
Kosmidis, Maria, et al. 2010. Journal of Immunotherapy (Hagerstown, Md.: 1997) 33 (5): 538-46. https://doi.org/10.1097/CJI.0b013e3181cc2615.
Krynitz, Britta, et al. 2013. International Journal of Cancer 132 (6): 1429-38. https://doi.org/10.1002/ijc.27765.
Lai, Chester, et al. 2016. Clinical Cancer Research: An Official Journal of the American Association for Cancer Research 22 (16): 4236-48. https://doi.org/10.1158/1078-0432.CCR-15-2614.
Lan, Cheng-Che E., et al. 2007. Journal of Dermatological Science 48 (3): 225-28. https://doi.org/10.1016/j.jdermsci.2007.08.003.
Laskin, Benjamin L., et al. 2017. Transplantation Direct 3 (8): e199. https://doi.org/10.1097/TXD.0000000000000715.
Lee, Kwai-Fong, et al. 2016. PLOS ONE 11 (5): e0155602. https://doi.org/10.13711journal.pone.0155602.
Lee, Peter K., et al. 2005. Dermatologic Surgery: Official Publication for American Society for Dermatologic Surgery [et Al.] 31 (6): 659-64. https://doi.org/10.1111/j.1524-4725.2005.31608.
Levi, F., et al. 1996. British Journal of Cancer 74 (11): 1847-50.
Lewis, Julia M., Bürgler, Christina D., Fraser, Juliet A. et al. 2015. Journal of Investigative Dermatology 135 (5): 1405-14. https://doi.org/10.1038/jid.2014.411.
Lewis, Julia M., Biirgler, Christina D., Freudzon, Marianna et al. 2015. Journal of Investigative Dermatology 135 (11): 2824-33. https://doi.org/10.1038/jid.2015.207.
Li, Wei-Hsuan, et al. 2012. Nephrology Dialysis Transplantation 27 (2): 833-39. https://doi.org/10.1093/ndt/gfr277.
Maimela, Nomathamsanqa Resegofetse, et al. 2018. Computational and Structural Biotechnology Journal 17 (November): 1-13. https://doi.org/10.1016/j.csbj.2018.11.004.
Manda, K. R., et al. 2016. Oncogene 35 (25): 3282-92. https://doi.org/10.1038/onc.2015.389.
Margolis, David J., et al. 2007. Dermatology 214 (4): 289-95. https://doi.org/10.1159/000100879.
Masuo, Takashige, et al. 2009. Cancer Letters 285 (1): 66-72. https://doi.org/10.1016/j.canlet.2009.05.001.
Medyouf, Hind, et al. 2007. Nature Medicine 13 (6): 736-41. https://doi.org/10.1038/nm1588.
Minami, Takashi, et al. 2013. Cell Reports 4 (4): 709-23. https://doi.org/10.1016/j.celrep.2013.07.021.
Ming, Mei, et al. 2015. Photochemistry and Photobiology 91 (1): 242-47. https://doi.org/10.1111/php.12318.
Modi, Badri G., et al. 2012. Science 335 (6064): 104-8. https://doi.org/10.1126/science.1211600.
Moloney, F. J., et al. 2006. British Journal of Dermatology 154 (3): 498-504. https://doi.org/10.1111/j.1365-2133.2005.07021.x.
Mühleisen, Beda, et al. 2009. Histopathology 55 (1): 67-76. https://doi.org/10.1111/j.1365-2559.2009.03324.x.
Nasti, Tahseen H., et al. 2011. Photochemistry and Photobiology 87 (2): 387-98. https://doi.org/10.1111/j.1751-1097.2010.00859.x.
Naylor, Mark, et al. 2005. Journal of Dermatological Treatment 16 (3): 149-53. https://doi.org/10.1080/09546630510041088.
Niwa, Y., et al. 2003. British Journal of Dermatology 149 (5): 960-67. https://doi.org/10.1111/j.1365-2133.2003.05735.x.
Ortner, Daniela, et al. 2017. Oncoimmunology 6 (2): e1260215. https://doi.org/10.1080/2162402X.2016.1260215.
Panhans-GroB, Andrea, et al. 2001. Journal of Allergy and Clinical Immunology 107 (2): 345-52. https://doi.org/10.1067/mai.2001.112600.
Penn, Israel. 2000. “Post-Transplant Malignancy: The Role Of Immunosuppression.” Drug Safety 23 (2): 101-13. https://doi.org/10.2165/00002018-200023020-00002.
Peuker, Kenneth, et al. 2016. Nature Medicine 22 (5): 506-15. https://doi.org/10.1038/nm.4072.
Philip, Mary, et al. 2017. Nature 545 (7655): 452-56. https://doi.org/10.1038/nature22367.
Piselli, Pierluca, et al. 2013. European Journal of Cancer 49 (2): 336-44. https://doi.org/10.1016/j.ejca.2012.09.013.
Quezada, Sergio A., et al. 2011. Immunological Reviews 241 (1): 104-18. https://doi.org/10.1111/j.1600-065X.2011.01007.x.
Ravindran, Anand, et al. 2014. Carcinogenesis 35 (4): 959-66. https://doi.org/10.1093/carcin/bgt486.
Rizvi, Syed Mohammad Husain, et al. 2017. JAMA Dermatology 153 (12): 1270-77. https://doi.org/10.1001/jamadermatol.2017.2984.
Roberts, Scott J., et al. 2007. Proceedings of the National Academy of Sciences of the United States of America 104 (16): 6770-75. https://doi.org/10.1073/pnas.0604982104.
Ruzicka, Thomas, et al. 1999. Archives of Dermatology 135 (5): 574-80. https://doi.org/10.100¹/archderm.135.5.574.
Sarvaria, Anushruti, et al. 2017. Cellular & Molecular Immunology 14 (8): 662-74. https://doi.org/10.1038/cmi.2017.35.
Schioppa, Tiziana, et al. 2011. Proceedings of the National Academy of Sciences 108 (26): 10662-67. https://doi.org/10.1073/pnas.1100994108.
Shang, Bin, et al. 2015. Scientific Reports 5 (October). https://doi.org/10.1038/srep15179.
Shoshan, Einav, et al. 2016. Cancer Research 76 (11): 3145-55. https://doi.org/10.1158/0008-5472.CAN-15-2511.
Siamakpour-Reihani, Sharareh, et al. 2011. PLoS ONE 6 (6). https://doi.org/10.1371/journal.pone.0020412.
Sigal, N H, and F J Dumont. 1992. Annual Review of Immunology 10 (1): 519-60. https://doi.org/10.1146/annurev.iy.10.040192.002511.
Staatz, Christine E., and Susan E. Tett. 2015. Clinical Pharmacokinetics 54 (10): 993-1025. https://doi.org/10.1007/s40262-015-0282-2.
Strobel, S. B., et al. 2018. The British Journal of Dermatology 179 (2): 405-12. https://doi.org/10.1111/bjd.16477.
Taylor, Nicholas A., et al. 2017. The Journal of Clinical Investigation 127 (9): 3472-83. https://doi.org/10.1172/JCI90499.
Tessari, G., et al. 2013. American Journal of Transplantation 13 (1): 214-21. https://doi.org/10.1111/j.1600-6143.2012.04294.x.
Thomson, A. W., et al. 1995. Therapeutic Drug Monitoring 17 (6): 584-591.
Tie, Xinxin, et al. 2013. PLoS ONE 8 (6). https://doi.org/10.1371/journal.pone.0066008.
Traitanon, Opas, et al. 2015. PLoS ONE 10 (6). https://doi.org/10.13711journalpone.0129658.
Tran Quang, C, et al. 2015. Cell Death & Disease 6 (2): e1658. https://doi.org/10.1038/cddis.2015.14.
Tripathi, P., et al. 2014. Oncogene 33 (14): 1840-49. https://doi.org/10.1038/onc.2013.132.
Urso, Katia, et al. 2019. Scientific Reports 9 (1): 1-14. https://doi.org/10.1038/s41598-019-45731-w.
Vajdic, Claire M., et al. 2006. JAMA 296 (23): 2823-31. https://doi.org/10.1001/jama.296.23.2823.
Villeneuve, P. J., et al. 2007. American Journal of Transplantation 7 (4): 941-48. https://doi.org/10.1111/j.1600-6143.2007.01736.x.
Walsh, Stephanie B., et al. 2011. Molecular Carcinogenesis 50 (7): 516-27. https://doi.org/10.1002/mc.20744.
Wang, Jennifer, et al. 2014. Journal of the American Academy of Dermatology 70 (4): 621-29. haps://doi.org/10.1016/j.jaad.2014.01.857.
Wang, Shuhuai, et al. 2012. Digestive Diseases and Sciences 57 (12): 3184-88. https://doi.org/10.1007/s10620-012-2255-8.
Wang, Zhen, et al. 2009. International Immunopharmacology, China Regulatory T Cells 2008, 9 (5): 564-69. https://doi.org/10.1016/j.intimp.2009.01.029.
Wheless, Lee, et al. 2014. Journal of the American Academy of Dermatology 71 (2): 359-65. https://doi.org/10.1016/j.jaad.2014.02.039.
Wollenberg, Andreas, et al. 2001. Journal of Allergy and Clinical Immunology 107 (3): 519-25. https://doi.org/10.1067/mai.2001.112942.
Wu, Xunwei, et al. 2010. Nature 465 (7296): 368-72. https://doi.org/10.1038/nature08996.
Wu, Xunwei, et al. 2018. PLOS Genetics 14 (5): e1007366. https://doi.org/10.1371/journal.pgen.1007366.
Xu, Wenwen, et al. 2016. Tumor Biology 37 (4): 4493-4500. https://doi.org/10.1007/s13277-015-4245-x.
Yusuf, Nabiha, et al. 2008. Cancer Research 68 (10): 3924-30. https://doi.org/10.1158/0008-5472.CAN-07-3059.
Zhang, Shah, et al. 2013. PLoS ONE 8 (5). https://doi.org/10.1371/journal.pone.0062154.

1. A method of preventing or treating skin cancer or skin precancer, the method comprising locally administering to the skin of a subject in need thereof an effective amount of a compound of Formula (I) or a prodrug thereof, 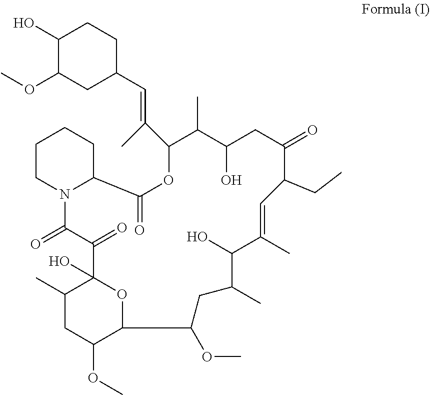 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]
wherein the subject is being administered an immunosuppressant agent that binds to FKBP12.

2. The method of claim 1, wherein the compound of Formula (I) is Compound 1:

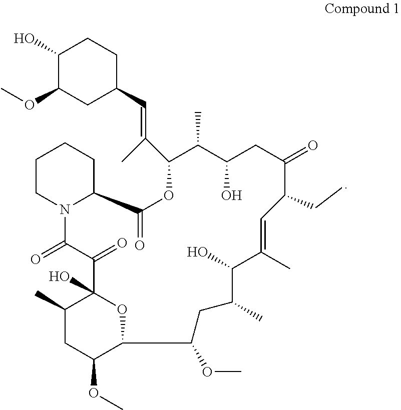 [SMILES: CC[C@@H]1/C=C(\C)[C@@H](O)[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

3. The method of claim 1, wherein substantially no compound of Formula (I), or prodrug thereof enters the blood stream after local administration to the skin of the subject.

4. The method of claim 1, wherein the subject is a human.

5. The method of claim 1, wherein the immunosuppressant agent that binds to FKBP12 forms a complex with FKBP12 and a further molecule.

6. The method of claim 1, wherein the immunosuppressant agent that binds to FKBP12 is selected from the group consisting of tacrolimus, sirolimus and everolimus.

7. The method of claim 6, wherein the immunosuppressant agent that binds to FKBP12 is tacrolimus.

8. The method of claim 1, wherein the subject is an organ transplant recipient.

9. The method of claim 8, wherein local administration of the compound of Formula (I), or prodrug thereof to the skin of the subject does not result in organ transplant rejection.

10. The method of claim 8, wherein the organ transplant recipient is being administered a combination of immunosuppressive agents which includes tacrolimus.

11. The method of claim 8, wherein the transplanted organ is selected from the group consisting of: liver, kidney, pancreas, heart, trachea, lung, face, intestine, eye, limb, cornea, bone and bone marrow.

12. The method of claim 1, wherein the method results in a reduction in the size or volume of a skin cancer or skin precancer, or results in eradication or elimination of a skin cancer or skin precancer.

13. The method of claim 1, wherein the skin cancer is a cutaneous squamous cell carcinoma (cSCC).

14. The method of claim 1, wherein the skin precancer is an actinic keratosis (AK), an intraepidermal carcinoma or Kaposi's sarcoma.

15. (canceled)

16. The method of claim 1, wherein the compound of Formula (I) or prodrug thereof is administered to the epidermis.

17. The method of claim 16, wherein the compound of Formula (I) or prodrug thereof is administered topically by rubbing or massaging a cream, ointment or salve containing the compound of Formula (I) or prodrug thereof onto the skin.

18. The method of claim 1, wherein the compound of Formula (I) or prodrug thereof is administered locally to the skin by intradermal injection.

19. (canceled)

20. A method of preventing or treating a skin condition, disorder or disease associated with administration of an immunosuppressant agent that binds to FKBP12, the method comprising locally administering to the skin of a subject in need thereof an effective amount of a compound of Formula (I) or a prodrug thereof, 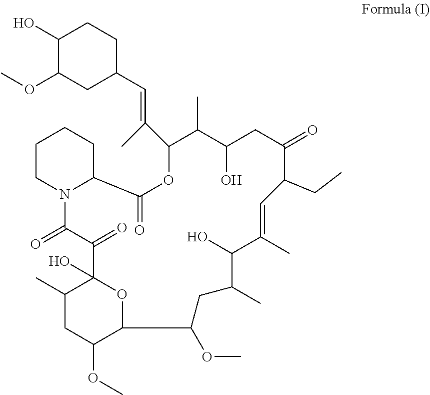 [SMILES: CCC1/C=C(\C)C(O)C(C)CC(OC)C2OC(O)(C(=O)C(=O)N3CCCCC3C(=O)OC(/C(C)=C/C3CCC(O)C(OC)C3)C(C)C(O)CC1=O)C(C)CC2OC]
wherein the subject is being administered an immunosuppressant agent that binds to FKBP12.

21. The method of claim 20, wherein the skin condition, disorder or disease is selected from the group consisting of: skin cancer, skin precancer, a fungal infection, a parasitic infection, a yeast infection, a viral infection, a bacterial infection, an inflammatory skin condition, a vascular skin condition and a benign skin lesion.

22. The method of claim 20, wherein the skin condition, disorder or disease is selected from the group consisting of: pruritis, folliculitis, onychopathy, a lesion or sore, poor wound healing, a rash, oedema, stomatitis, hair loss and hypertrichosis.
